(19) United States
(12) Patent Application Publication
(10) Pub. No.: US 2026/0235628 A1
(43) Pub. Date: Aug. 13, 2026

(54) U-p53 PEPTIDES AS MARKERS IN THE RATE OF PROGRESSION OF COGNITIVE DECLINE TO ALZHEIMER'S DISEASE

(71) Applicant: Diadem SpA, Milan (IT)

(72) Inventors: Simona Piccirella, Milan (IT); Paul Kinnon, Milan (IT)

(21) Appl. No.: 19/261,264

(22) Filed: Jul. 7, 2025

Related U.S. Application Data
(63) Continuation of application No. PCT/US24/10696, filed on Jan. 8, 2024.
(60) Provisional application No. 63/478,973, filed on Jan. 8, 2023.

(51) Int. Cl. G01N 33/68; A23L 33/00; A61K 31/05; A61K 31/07; A61K 31/12; A61K 31/122; A61K 31/13; A61K 31/192; A61K 31/196; A61K 31/202; A61K 31/205; A61K 31/325; A61K 31/355; A61K 31/37; A61K 31/375; A61K 31/40; A61K 31/4045; A61K 31/405; A61K 31/407; A61K 31/421; A61K 31/445; A61K 31/519; A61K 31/5415; A61K 31/55; A61K 31/616; A61K 31/635; A61K 39/00; A61K 45/06; A61P 25/28; C07K 16/18

(52) U.S. Cl. CPC G01N 33/6896; A23L 33/30; A61K 31/05; A61K 31/07; A61K 31/12; A61K 31/122; A61K 31/13; A61K 31/192; A61K 31/196; A61K 31/202; A61K 31/205; A61K 31/325; A61K 31/355; A61K 31/37; A61K 31/375; A61K 31/40; A61K 31/4045; A61K 31/405; A61K 31/407; A61K 31/421; A61K 31/445; A61K 31/519; A61K 31/5415; A61K 31/55; A61K 31/616; A61K 31/635; A61K 45/06; A61P 25/28; C07K 16/18; G01N 33/6848; A61K 2039/505; C07K 2317/21; C07K 2317/24; G01N 2333/4748; G01N 2800/2821; G01N 2800/50; G01N 2800/52

(57) ABSTRACT

U-p53 peptide P1 is useful in the determination of the rate of progression of Alzheimer's disease (AD). By quantitating the level of U-p53 peptides in a subject's biological sample, the rate of progression of Alzheimer's disease at the pre-clinical and prodromal stages of the disease in a subject can be determined.

### RELATED APPLICATIONS

This application is a continuation application of International Patent Application No. PCT/US24/10696, filed Jan. 8, 2024, which claims priority to U.S. Provisional Application No. 63/478,973 filed on Jan. 8, 2023, the contents of each of which is herein incorporated by reference in its entirety.

### FIELD OF THE INVENTION

The present invention refers to the use of U-p53 peptides in the determination of rate of progression of Alzheimer's disease (AD) in a subject. The invention also provides for a diagnostic method based on a highly accurate mass spectrometry analysis for determination of rate of progression of Alzheimer's disease in a subject, by quantitating the level of the U-p53 peptides.

### SEQUENCE LISTING

This application contains a Sequence Listing which has been submitted electronically as a WIPO Standard ST.26 XML file via Patent Center, created on Jul. 7, 2025, is entitled “121689-10503.xml” and is 23,776 bytes in size. The sequence listing is incorporated herein by reference in its entirety.

### BACKGROUND

Alzheimer's disease (AD) is a progressive neurodegenerative disorder, starting with an asymptomatic phase of normal cognition lasting approximately two decades. While some cognitive individuals (CN) experience subjective memory complaints (SMC) and some have no memory complaints (NMC), all eventually progress to mild cognitive impairment (MCI) before finally reaching the AD stage. To date, the formal diagnosis of AD dementia, as stated by the National Institute on Aging and Alzheimer's Association (NIA-AA), relies on neuropsychological tests further confirmed by brain imaging and cerebrospinal fluid (CSF) sampling.

The confirmation of the presence of a large amount of altered conformational p53 isoform (U-p53) as an early risk factor for AD have been demonstrated in different published studies [1-3]. Initially, more than 400 subjects among AD, Mild Cognitive Impairment, Parkinson Disease, other Dementia and healthy subjects were enrolled in different independent studies and tested for Unfolded p53 by using different techniques (immunoprecipitation experiments, FACS analysis, ELISA) with a commercial conformational specific anti-p53 antibody [4-7].

In 2006 Uberti et al. [8], demonstrated that fibroblasts from sporadic Alzheimer's disease (AD) patients specifically expressed an anomalous and detectable conformational state of p53 that differentiate these cells from fibroblasts of age-matched non-AD subjects. In this conformational altered state, p53 lost its ability to transactivate its target genes, and consequently its biological functions [9-10]. The higher amount of unfolded p53 (U-p53) was also confirmed in blood of AD compared to healthy-non demented subjects or patients affected by other dementia and Parkinson's disease (PD), as well as in Mild cognitive impairment (MCI) converted to AD.

EP3201234B1 discloses the development of a new conformational specific anti-U-p53 antibody named 2D3A8, that binds to an epitope (aa 282-297), accessible only when p53 loses its wild type conformation towards an unfolded phenotype. Unlike the pre-existing commercial p53-antibody (PAb240, aa214-217), the 2D3A8 antibody showed higher sensitivity and specificity in identifying AD patients compared to healthy elderly in Oviedo cohort.

WO2020178620 disclosed that U-p53 peptides such as P1, P2, P3 and P5 can be used in the diagnosis and/or prognosis of Alzheimer's disease (AD) in a biological sample. The WO2020178620 disclosure also provides a method for the diagnosis and/or prognosis of Alzheimer's disease based on the presence of U-p53 peptides in a biological sample.

WO2022023964 disclosed that post translational modifications (PTM) of unfolded p53 protein can be used in the diagnosis of neurodegenerative disease and cognitive decline and/or in the prognosis of Alzheimer's disease at different stages and/or of neurodegenerative disease in a biological sample.

### SUMMARY OF THE INVENTION

The present invention relates to a diagnostic method based on the identification and quantification of the U-p53 peptide P1, wherein detection of a given femtomole (fm) amount of peptide P1 permits determination of the rate and therefore the timing of progression into Alzheimer's disease.

The disclosure provides for identification of a subject with respect to the subject's risk of progression to Alzheimer's disease using a simple biological assay that detects a specific and precise amount of peptide P1, which is an in vitro generated proteolytic peptide of U-p53 protein. The subject's risk of progression is classified according to the invention as high, moderate or slow, depending on the precise amount of the one or more proteolytic peptides of U-p53 are detected in the sample.

The invention is based on in vitro quantitation of U-p53 peptide P1, wherein quantitation of at least 0.9 femtomoles indicates high risk for progression to Alzheimer' disease in the subject, quantitation of at least 0.75 to less than 0.9 femtomoles of P1 indicates moderate progression (i.e., excludes high risk of progression) to Alzheimer' disease in the subject, and quantitation of less than 0.75 femtomoles of P1 indicates slow progression (i.e., low risk of progression) to Alzheimer' disease in the subject.

In one aspect, the disclosure provides a method of classifying a subject as high risk or low risk for progression to Alzheimer's disease, the method comprising subjecting isolated U-p53 protein to protease digestion to generate one or more proteolytic peptides, wherein one or more proteolytic peptides comprises peptide P1 having SEQ ID NO: 1 TEEENLR; and quantifying an amount of P1 peptide in a reaction mixture, wherein at least 0.9 femtomoles of P1 in the reaction mixture indicates high risk progression to Alzheimer' disease in the subject, wherein at least 0.75 but less than 0.9 femtomoles of P1 in the reaction mixture indicates moderate progression or excludes high risk for progression to Alzheimer' disease in the subject, and wherein less than 0.75 femtomoles of P1 in the reaction mixture indicates low risk for progression or slow progression to Alzheimer' disease in the subject, thereby classifying the subject.

In another aspect, the disclosure provides a method of classifying a subject as a high risk progressor of Alzheimer's disease, the method comprising subjecting a biological sample from the subject to immunoprecipitation using an antibody specific for p53 protein to provide isolated U-p53 protein; subjecting the isolated U-p53 protein to protease digestion to generate one or more proteolytic peptides, wherein the one or more proteolytic peptides comprises peptide P1 having SEQ ID NO: 1 TEEENLR; and quantifying an amount of at least 0.9 femtomoles of P1 peptide in a reaction mixture, wherein at least 0.9 femtomoles of P1 in the reaction mixture indicates high risk for progression to Alzheimer' disease in the subject, wherein the antibody specific for U-p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15).

In another aspect, the disclosure provides a method of classifying a subject as a moderate progressor or excluding high risk of progression of Alzheimer's disease, the method comprising subjecting a biological sample from the subject to immunoprecipitation using an antibody specific for U-p53 protein to provide isolated U-p53 protein; subjecting the isolated U-p53 protein to protease digestion to generate one or more proteolytic peptides, wherein the one or more proteolytic peptides comprises peptide P1 having SEQ ID NO: 1 TEEENLR; and quantifying an amount of at least 0.75 but less than 0.9 femtomoles of P1 peptide in a reaction mixture, wherein at least 0.75 but less than 0.9 femtomoles of P1 in the reaction mixture indicates moderate progression or excludes high risk of progression to Alzheimer' disease in the subject, wherein the antibody specific for U-p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO: 13), CDR2 (SEQ ID NO: 14) and CDR3 (SEQ ID NO: 15).

In another aspect, the disclosure provides a method of classifying a subject as a slow progressor of Alzheimer's disease, the method comprising subjecting a biological sample from the subject to immunoprecipitation using an antibody specific for p53 protein to provide isolated U-p53 protein; subjecting the isolated U-p53 protein to protease digestion to generate one or more proteolytic peptides, wherein the one or more proteolytic peptides comprises peptide P1 having SEQ ID NO: 1 TEEENLR; and quantifying an amount of less than 0.75 femtomoles of P1 peptide in a reaction mixture, wherein less than 0.75 femtomoles of P1 in the reaction mixture indicates slow progression or low risk of progression to Alzheimer' disease in the subject, wherein the antibody specific for p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO: 13), CDR2 (SEQ ID NO: 14) and CDR3 (SEQ ID NO: 15).

In another aspect, the disclosure provides a method of classifying a subject as a high risk progressor to Alzheimer's disease, the method comprising subjecting isolated unfolded p53 (U-p53) protein to proteolytic digestion to generate one or more peptides comprising P1 and quantitating the amount of P1 in the sample, wherein at least 0.9 femtomoles of the P1 in the reaction mixture indicates progression to Alzheimer' disease in the subject within six years.

In some embodiments, P1 comprises the sequence of: TEEENLR (SEQ ID NO: 1). In some embodiments, P1 consists essentially of SEQ ID NO:1. In some embodiments, P1 consists of SEQ ID NO: 1. In some embodiments, P1 is SEQ ID NO: 1.

In some embodiments, the isolated U-p53 is obtained by subjecting the biological sample from the subject to immunoprecipitation using an antibody specific for unfolded p53 (U-p53) protein.

In some embodiments, the antibody comprises a heavy chain (SEQ ID NO: 6) and a light chain (SEQ ID NO: 7). In some embodiments, the antibody specific for the unfolded p53 protein (U-p53) comprises a heavy chain variable region (SEQ ID NO: 8) and a light chain variable region (SEQ ID NO:9).

In some embodiments, the antibody specific for the unfolded p53 protein (U-p53) comprises a heavy chain variable region comprising CDR1 (SEQ ID NO:10), CDR2 (SEQ ID NO: 11) and CDR3 (SEQ ID NO: 12). In some embodiments, the antibody specific for the unfolded p53 protein (U-p53) comprises a light chain variable region comprising CDR1 (SEQ ID NO:13), CDR2 (SEQ ID NO: 14) and CDR3 (SEQ ID NO: 15).

In some embodiments, the antibody specific for the unfolded p53 protein (U-p53) comprises a heavy chain variable region (SEQ ID NO: 8) comprising CDR1 (SEQ ID NO: 10), CDR2 (SEQ ID NO: 11) and CDR3 (SEQ ID NO: 12) and a light chain variable region (SEQ ID NO:9) comprising CDR1 (SEQ ID NO:13), CDR2 (SEQ ID NO: 14) and CDR3 (SEQ ID NO: 15).

In some embodiments of any of the aforesaid methods disclosed herein, the presence of at least 1.06 femtomoles of P1 peptide in the reaction mixture indicates rapid progression of Alzheimer's disease in the subject. In some embodiments, the progression to Alzheimer's disease occurs within two years of testing.

In some embodiments of any of the aforesaid methods disclosed herein, the presence of at least 0.90 femtomoles but less than 1.06 femtomoles of P1 peptide in the reaction mixture indicates high risk of progression of Alzheimer's disease in the subject. In some embodiments, the progression to Alzheimer's disease occurs within six years of testing.

In some embodiments of any of the aforesaid methods disclosed herein, the quantifying is performed through using mass spectrometry analysis. In some embodiments of any of the aforesaid methods, the mass spectrometry analysis comprises HPLC-mass spectrometry. In some embodiments of any of the aforesaid methods, the mass spectrometry analysis comprises LC-MS/MS spectrometry. In some embodiments, the quantitation of the amount of U-p53 P1 peptide is performed by comparing the same with a given internal control.

In some embodiments, the quantitation of the amount of U-p53 P1 peptide is done by comparing the same with a known standard. In some embodiments, the internal control is a peptide internal control.

In some embodiments of any of the aforesaid methods, the peptide control is a labelled peptide. In some embodiments of any of the aforesaid methods, the labelled peptide control is internal to the reaction mixture and is represented as a value comprising its concentration in the reaction mixture.

In some embodiments of any of the aforesaid methods, the quantifying is performed using Selected Reaction Monitoring (SRM). In some embodiments of any of the aforesaid methods, the quantifying is performed using Selected Reaction Monitoring (SRM) and the SRM identifies and quantifies the P1 peptide in the reaction mixture by comparing the same with the peptide control value. In some embodiments of any of the aforesaid methods, the biological sample is one of blood, serum, plasma, saliva and cerebrospinal fluid (CSF). In some embodiments of any of the aforesaid methods biological sample is plasma.

In some embodiments of any of the aforesaid methods, the biological sample is subjected to protein plasma depletion prior to the immunoprecipitation.

In some embodiments of any of the aforesaid methods, when the P1 peptide in the reaction mixture is at least 1.06 femtomoles, preferably at least 1.1 femtomoles, the subject is a rapid progressor who will progress to AD within 2 years of testing.

In some embodiments of any of the aforesaid methods, the protein plasma depletion is accomplished by one or more of: HPLC, a chromatographic column, and/or chemical treatment of the biological sample. In some embodiments, the method provides for the diagnosis of rate of progression of Alzheimer's disease in an asymptomatic subject or in a subject exhibiting mild cognitive impairment.

In some embodiments, the method provides for prognosis of Alzheimer's disease within 2-6 years in an asymptomatic subject or a subject exhibiting mild cognitive impairment by quantitating the amount of U-p53-P1 proteolytic peptide present in the reaction mixture obtained from the biological sample of the subject, wherein at least 0.9 femtomoles of the P1 in the reaction mixture indicates that the subject will progress to AD within 2-6 years

In some embodiments, the isolated U-p53 is obtained by subjecting the biological sample from the subject to immunoprecipitation using an antibody specific for U-p53 protein. The antibody specific for U-p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15).

In some embodiments of any of the aforesaid methods, the antibody comprises a heavy chain variable region having SEQ ID NO: 8 and a light chain variable region having SEQ ID NO: 9. In some embodiments of any of the aforesaid methods, the antibody comprises a heavy chain having SEQ ID NO: 6 and a light chain having SEQ ID NO: 7.

In another aspect, the disclosure provides a method of classifying a subject as a moderate progressor to Alzheimer's disease, the method comprising subjecting isolated unfolded p53 (U-p53) protein to proteolytic digestion to generate one or more peptides comprising P1 and quantitating the amount of P1 in the sample, wherein 0.75 to less than 0.9 femtomoles of the P1 in the reaction mixture indicates moderate progression to Alzheimer's disease in the subject.

In some embodiments of any of the aforesaid methods disclosed herein, the quantifying is performed through using mass spectrometry analysis. In some embodiments of any of the aforesaid methods, the mass spectrometry analysis comprises HPLC-mass spectrometry. In some embodiments of any of the aforesaid methods, the mass spectrometry analysis comprises LC-MS/MS spectrometry. In some embodiments, the quantitation of the amount of U-p53 P1 peptide is done by comparing the same with a given internal control. In some embodiments, the quantitation of the amount of U-p53 P1 peptide is done by comparing the same with a known standard. In some embodiments, the internal control is a peptide internal control. In some embodiments of any of the aforesaid methods, the peptide control is a labelled peptide. In some embodiments of any of the aforesaid methods, the labelled peptide control is internal to the reaction mixture and is represented as a value comprising its concentration in the reaction mixture.

In some embodiments of any of the aforesaid methods, the quantifying is performed using Selected Reaction Monitoring (SRM). In some embodiments of any of the aforesaid methods, the quantifying is performed using Selected Reaction Monitoring (SRM) and the SRM identifies and quantifies the P1 peptide in the reaction mixture by comparing the same with the peptide control value. In some embodiments of any of the aforesaid methods, the biological sample is one of blood, serum, plasma, saliva, and cerebrospinal fluid (CSF). In some embodiments of any of the aforesaid methods, the biological sample is plasma.

In some embodiments of any of the aforesaid methods, the biological sample is subjected to protein plasma depletion prior to the immunoprecipitation. In some embodiments of any of the aforesaid methods, the protein plasma depletion is accomplished by one or more of: HPLC, a chromatographic column, and/or chemical treatment of the biological sample. In some embodiments, the method provides for the diagnosis of rate of progression of Alzheimer's disease in an asymptomatic subject or in a subject exhibiting mild cognitive impairment.

In some embodiments, the isolated U-p53 is obtained by subjecting a biological sample from the subject to immunoprecipitation using an antibody specific for U-p53 protein to provide isolated unfolded p53 protein (U-p53), and, wherein the antibody specific for U-p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15).

In some embodiments of any of the aforesaid methods, the antibody comprises a heavy chain variable region having SEQ ID NO: 8 and a light chain variable region having SEQ ID NO: 9. In some embodiments of any of the aforesaid methods, the antibody comprises a heavy chain having SEQ ID NO: 6 and a light chain having SEQ ID NO: 7.

In another aspect, the disclosure provides a method of classifying a subject as a slow progressor to Alzheimer's disease, the method comprising: subjecting isolated unfolded p53 (Up53) protein to proteolytic digestion to generate one or more peptides comprising P1 and quantitating the amount of P1 in the sample, wherein less than 0.75 femtomoles of the P1 in the reaction mixture indicates slow progression or low risk of progression to Alzheimer' disease in the subject.

In some embodiments of any of the aforesaid methods disclosed herein, the quantifying is performed through using mass spectrometry analysis. In some embodiments of any of the aforesaid methods, the mass spectrometry analysis comprises HPLC-mass spectrometry. In some embodiments of any of the aforesaid methods, the mass spectrometry analysis comprises LC-MS/MS spectrometry. In some embodiments, the quantitation of the amount of U-p53 P1 peptide is done by comparing the same with a given internal control. In some embodiments, the quantitation of the amount of U-p53 P1 peptide is done by comparing the same with a known standard. In some embodiments, the internal control is a peptide internal control. In some embodiments of any of the aforesaid methods, the peptide control is a labelled peptide. In some embodiments of any of the aforesaid methods, the labelled peptide control is internal to the reaction mixture and is represented as a value comprising its concentration in the reaction mixture.

In some embodiments of any of the aforesaid methods, the quantifying is performed using Selected Reaction Monitoring (SRM) In some embodiments of any of the aforesaid methods the quantifying is performed using Selected Reaction Monitoring (SRM) and the SRM identifies and quantifies the P1 peptide in the reaction mixture by comparing the same with the peptide control value. In some embodiments of any of the aforesaid methods the biological sample is one of blood, serum, plasma, saliva, and cerebrospinal fluid (CSF). In some embodiments of any of the aforesaid methods biological sample is plasma.

In some embodiments of any of the aforesaid methods, the biological sample is subjected to protein plasma depletion prior to the immunoprecipitation. In some embodiments of any of the aforesaid methods the protein plasma depletion is accomplished by one or more of: HPLC, a chromatographic column, and/or chemical treatment of the biological sample. In some embodiments, the method provides for the diagnosis of rate of progression of Alzheimer's disease in an asymptomatic subject or in a subject exhibiting mild cognitive impairment.

In some embodiments, the isolated U-p53 is obtained by subjecting a biological sample from the subject to immunoprecipitation using an antibody specific for U-p53 protein to provide isolated unfolded p53 protein (U-p53), and, wherein the antibody specific for U-p53 protein comprises a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15).

In some embodiments of any of the aforesaid methods, the antibody comprises a heavy chain variable region having SEQ ID NO: 8 and a light chain variable region having SEQ ID NO: 9. In some embodiments of any of the aforesaid methods the antibody comprises a heavy chain having SEQ ID NO: 6 and a light chain having SEQ ID NO: 7.

In some embodiments of any of the aforesaid methods, the method further comprising the step of treating the subject with a disease-modifying medication that reduces amyloid plaques such as aducanumab and lecanemab.

In some embodiments of any of the aforesaid methods, the method further comprising the step of treating the subject with a drug selected from the group consisting of memantine galantamine, rivastigmine, and donepezil.

In some embodiments of any of the aforesaid methods, the method further comprising the step of treating the subject with one or more of MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

In some embodiments of any of the aforesaid methods, the vitamin/antioxidant supplement is one or more of vitamin A, vitamin B, folic acid, vitamin C, vitamin D, vitamin E, DHA (docosahexaenoic acid), ubiquinone, lycopene, coenzyme Q10 and ellagic acid, ascorbic acid, masoprocol, pramipexole, nitric oxide, allopurinol, pentoxifylline, melatonin, probucol, quercetin, acetylcysteine, n acetylcysteine, acetyl-L-carnitine and 1-methylfolate and resveratrol.

In some embodiments of any of the aforesaid methods, the blood pressure reducing medication is selected from the group consisting of Angiotensin-converting enzyme (ACE) inhibitors, Angiotensin receptor blockers (ARBs), Calcium-channel blockers and Beta-blockers.

In some embodiments of any of the aforesaid methods, the anti-inflammatory medication is selected from the group consisting of Aspirin, Diclofenac, Etodolac, Fenoprofen, Flurbiprofen, Ibuprofen, Indomethacin, Meclofenamate, Mefenamic Acid, Nabumetone, Naproxen, Oxaprozin, Piroxicam, Sulindac, Tolmetin, Celecoxib and Meloxicam.

The characteristics and the advantages of the present invention will become apparent from the following detailed description and the working examples provided for illustrative purposes.

### DETAILED DESCRIPTION OF THE INVENTION

In one aspect, the disclosure relates to a highly accurate method for the determination of the rate of progression of Alzheimer's disease in a subject. The rate of progression refers to the time period within which a subject will progress into AD.

In some embodiments, the subject is a rapid progressor and will progress or more likely to progress to AD within 2 years of testing. In some embodiments, the subject is a high risk progressor will progress or more likely to progress to AD within 6 years of testing. In some embodiments, the subject is a low risk progressor and will not or less likely to progress to AD within 2 years of testing. In some embodiments, the subject is a low risk progressor and will not or less likely to progress to AD within 6 years of testing. In some embodiments, the subject exhibits moderate risk of progression to AD within 6 years. The time frame of progression is dependent on the amount of U-p3-P1 peptide.

In one aspect, the disclosure relates to quantitation of U-p53 peptides for the determination of the rate of progression of Alzheimer's disease in a subject. In some embodiments, the subject is cognitively normal with no symptoms. In some embodiments, the subject has minimal symptoms and/or mild cognitive impairment. The methods are based on the identification and quantification of the level of the U-p53 peptide P1: TEEENLR (SEQ ID NO: 1). Other peptides that may be generated by proteolytic digestion of U-p53 protein include P2: TEEENLRK[GG]K (SEQ ID NO: 2), P3: KKPLDGEYFTLQIR (SEQ ID NO: 3), P4: EPGGSRAHSSHLK (SEQ ID NO: 4) and P5: GEPHHELPPGSTKRALPNNTSSSPQPK (SEQ ID NO: 5).

The method is advantageously fast, requires a small volume of biological sample and quantifies the concentration of U-p53 peptide P1 in a sample analysed.

In addition, the method allows the selection and/or stratification of subjects for clinical trials by differentiating patients that will experience rapid progression to AD from others who will experience moderate or slow progression to AD to ensure that the drug being tested in the trial is administered to a uniform cohort of patients.

Furthermore, the methods can be used in classifying subjects into those who are at high risk of progressing into Alzheimer's disease and those who are at moderate or low risk of progressing into Alzheimer's disease.

Furthermore, the methods can be used in classifying subjects who are a given age, for example subjects over the age of 40, 50, 60 or 70 years into those who are at high risk of progressing into Alzheimer's disease and those who are at moderate or low risk of progressing into Alzheimer's disease.

In another aspect, the disclosure relates to a method of treating a subject suffering from the progression of Alzheimer's disease. Depending upon the rate of progression of AD, the subject is categorized as high risk progressor, moderate or excluding high risk progressor, slow or low risk progressor. Once the subject is categorized, a medical professional can provide tailored treatment for each category. The method relates to quantitation of U-p53 peptides for the determination of the rate of progression of Alzheimer's disease in a subject. In some embodiments, the subject is cognitively normal with no symptoms. In some embodiments, the subject has minimal symptoms and/or mild cognitive impairment. In some embodiments, the subjects categorized as high risk progressors are treated with disease modifying medications. In some embodiments, the subjects categorized as moderate risk or excluding high risk progressors are treated with disease modifying medications. In some embodiments, the subjects categorized as slow progressors or low risk progressors are treated with specific diets and/or supplements.

An embodiment of the present invention is the use of the U-p53 peptide P1 (SEQ ID NO: 1; TEEENLR) as in vitro biomarker for the diagnosis of rate of progression of Alzheimer's disease. In some embodiments, the U-p53 P1 peptide identified has an amino acid sequence that contains all seven amino acids TEEENLR and may also contain one or two additional amino acids on either end of the peptide.

A further embodiment of the present invention is an in vitro or ex vivo method for the determination of rate of progression of Alzheimer's disease, the method comprising the steps of quantifying peptide P1 in a biological sample of a subject by comparing the same with a given internal control.

In some embodiments, the method of the present invention comprises detecting less than 0.75 fm or detecting at least 0.75 fm and less than 0.9 fm, or detecting at least 0.9 fm, or detection at least 1.06 fm, preferably at least 1.1 femtomoles, of P1 peptide in the reaction mixture. By detecting one of these recited specific amounts of P1, one of skill in the art is then able to determine the rate of progression of Alzheimer's disease in a patient.

In some embodiments, the method according to the present invention, isolated U-p53 is obtained by immunoprecipitating the biological sample with a monoclonal antibody that binds to U-p53 protein. Preferably, the monoclonal antibody is the antibody 2D3A8.

In some embodiments, the isolated U-p53 protein is subjected to proteolysis to generate the reaction mixture. In some embodiments, the reaction mixture thus generated comprises one or more U-p53 peptides, P1, P2, P3, P4 and P5.

In some embodiments, the one or more U-p53 peptides are detected using mass spectrometry or a similar method to analyze a reaction mixture after proteolytic digestion of U-p53 protein isolated from human plasma of a patient that may or may not exhibit one or more symptoms of cognitive impairment, dementia or Alzheimer's disease, or a patient exhibiting no or few symptoms but having a family history that can predispose to the development of AD.

In some embodiments, the methods are performed on a subject that may be at a pre-clinical or a prodromal clinical stage of Alzheimer's, an MCI stable patient, and a cognitive normal subject. In some embodiments, the level of one or more U-p53 peptides may be quantitated in a reaction mixture using a highly sensitive assay such as selective reaction monitoring (SRM) mass spectrometry method carrying out an Area Under the ROC Curve (AUC) (where ‘ROC Curve’ means ‘receiver operating characteristic curve’). In some embodiments, the quantitation of U-p53 peptides is done using Liquid Chromatography with tandem mass spectrometry (LC-MS-MS). In some embodiments of the method according to the present invention uses mass spectrometry analysis, preferably by HPLC-mass spectrometry.

In some embodiments of the method, labelled peptides such as labelled P1 or labelled P2 or labelled P3 or labelled P4 are used as internal controls for quantitation of U-p53 peptides in the reaction mixture. In some embodiments, the reaction mixture is spiked with a known amount of labelled peptide P1 in order to quantitate the amount of P1 peptide generated by the proteolytic digestion of isolated U-p53 protein.

In some embodiments, the in vitro or ex vivo method of the present invention comprises the following steps:
providing a biological sample;
performing protein immunoprecipitation by an antibody that binds a U-p53 peptide;
performing protein fragmentation by trypsin;
performing Strong Cation Exchange Chromatography of the peptides;
and
quantifying the U-p53 peptide by comparing the same with a given internal control, by:
performing a Selected Reaction Monitoring analysis to identify and quantify the U-p53 peptide by comparing it with a given control.

Preferably, the antibody of aforesaid step is 2D3A8.

In some embodiments, the biological sample of is subjected to protein plasma depletion by HPLC or chromatographic columns, before performing immunoprecipitation.

In some embodiments, the in vitro or ex vivo method of the present invention is used for the determination of rate of progression of Alzheimer's disease in an asymptomatic individual and/or a subject suffering from MCI.

In some embodiments, the U-p53 protein is isolated from the sample using a ligand or an aptamer that is specific to U-p5 protein instead of an antibody that is specific to U-p53 protein. Aptamers are short, single-stranded DNA or RNA (ssDNA or ssRNA) molecules that can selectively bind to a specific target such as U-p53 protein. Aptamers assume a variety of shapes due to their tendency to form helices and single-stranded loops. Aptamers with affinity for a desired target are selected from a large oligonucleotide library through a process called SELEX, which stands for Sequential Evolution of Ligands by Exponential Enrichment. Through an iterative process, non-binding aptamers are discarded and aptamers binding to the proposed target are expanded. Initial positive selection rounds are sometimes followed by negative selection. This improves the selectivity of the resulting aptamer candidates. Multiple rounds of SELEX are performed with increasing stringency to enhance enrichment of the oligonucleotide pool. The aptamer thus selected is specific for U-p53 protein and can be used to isolate the U-p53 protein from the biological sample for further analysis such as protease digestion, mass spectrometry and quantitation of P1 peptide. (Keefe, A., Pai, S. & Ellington, A. Aptamers as therapeutics. Nat Rev Drug Discov 9, 537-550 (2010).

According to a further embodiment, the in vitro or ex vivo method of the present invention is used for the determination of rate of progression of Alzheimer's disease in an asymptomatic individual and/or a subject suffering from MCI.

In some embodiments, the asymptomatic and MCI subjects that have moderate risk of progression of Alzheimer's dementia, the quantity of the U-p53 peptide is of 0.75 fmol/10 μl.

In some embodiments, the asymptomatic and MCI subjects that have high risk of progression of Alzheimer's dementia within six years, the quantity of the U-p53 peptide is at least 0.9 fmol/10 μl.

In some embodiments, the asymptomatic and MCI subjects are rapid progressors that have high risk of progression of Alzheimer's dementia within two years, the quantity of the U-p53 peptide is at least 1.06 fmol/10 μl. In some embodiments, the quantity of the U-p53 peptide is at least 1.1 fmol/10 μl.

In some embodiments, the area under the receiver operating characteristic curve (AUC) in an asymptomatic individual is at least 80%.

In some embodiments, the area under the receiver operating characteristic curve (AUC) in an asymptomatic individual is at least 90%.

Measures of accuracy of a diagnostic/prognostic method include sensitivity and specificity. Sensitivity is the probability of a positive test result among those having the target condition, and it is measured by the formula: Sensitivity=true positives/(true positive+false negative). Specificity is the probability of a negative test result among those without the target condition, and it is measured by the formula: Specificity=true negatives/(true negative+false positives).

In some embodiments, the sensitivity of detection of progression of AD using the U-p53 peptide P1 is at least 70% in an asymptomatic individual. In some embodiments, the sensitivity of detection of progression of AD using the U-p53 peptide P1 is at least 75% in an asymptomatic individual. In some embodiments the sensitivity of detection of progression of AD using the U-p53 peptide P1 is at least 80% in an asymptomatic individual.

In some embodiments, the sensitivity of detection of progression of AD using the U-p53 peptide P1 is at least 85% in an asymptomatic individual. In some embodiments, the sensitivity of detection of progression of AD using the U-p53 peptide P1 is at least 90% in an asymptomatic individual. In some embodiments, the sensitivity of detection of progression of AD using the U-p53 peptide P1 is at least 92% in an asymptomatic individual.

In some embodiments, the sensitivity of detection of progression of AD using the U-p53 peptide P1 is at least 94% in an asymptomatic individual. In some embodiments, the sensitivity of detection of progression of AD using the U-p53 peptide P1 is at least 95% in an asymptomatic individual. In some embodiments, the sensitivity of detection of progression of AD using the U-p53 peptide P1 is at least 96% in an asymptomatic individual.

In some embodiments, the sensitivity of detection of progression of AD using the U-p53 peptide P1 is at least 97% in an asymptomatic individual. Preferably, in an asymptomatic individual, the sensitivity of detection of progression of AD using the U-p53 peptide P1 is at least 98%.

In some embodiments, the sensitivity of detection of progression of AD using the U-p53 peptide P1 is at least 70% in a subject suffering from MCI. In some embodiments, the sensitivity of detection of progression of AD using the U-p53 peptide P1 is at least 75% in a subject suffering from MCI. In some embodiments, the sensitivity of detection of progression of AD using the U-p53 peptide is at least 80% in a subject suffering from MCI. In some embodiments, the sensitivity of detection of progression of AD using the U-p53 peptide P1 is at least 85% in a subject suffering from MCI. In some embodiments, the sensitivity of detection of progression of AD using the U-p53 peptide P1 is at least 90% in a subject suffering from MCI. In some embodiments, the sensitivity of detection of progression of AD using the U-p53 peptide P1 is at least 92% in a subject suffering from MCI.

In some embodiments, the sensitivity of detection of progression of AD using the U-p53 peptide P1 is at least 94% in a subject suffering from MCI. In some embodiments, the sensitivity of detection of progression of AD using the U-p53 peptide P1 is at least 95% in a subject suffering from MCI. In some embodiments, the sensitivity of detection of progression of AD using the U-p53 peptide P1 is at least 96% in a subject suffering from MCI.

In some embodiments, the sensitivity of detection of progression of AD using the U-p53 peptide P1 is at least 97% in a subject suffering from MCI. Preferably, the sensitivity of detection of progression of AD using the U-p53 peptide P1 is at least 98% in a subject suffering from MCI.

In some embodiments, the specificity of detection of progression of AD using the U-p53 peptide P1 is at least 70% in an asymptomatic individual. In some embodiments, the specificity of detection of progression of AD using the U-p53 peptide P1 is at least 75% in an asymptomatic individual. In some embodiments, the specificity of detection of progression of AD using the U-p53 peptide P1 is at least 80% in an asymptomatic individual. In some embodiments, the specificity of detection of progression of AD using the U-p53 peptide P1 is at least 85% in an asymptomatic individual. In some embodiments, the specificity of detection of progression of AD using the U-p53 peptide P1 is at least 90% in an asymptomatic individual. In some embodiments, the specificity of detection of progression of AD using the U-p53 peptide P1 is at least 92% in an asymptomatic individual. In some embodiments, the specificity of detection of progression of AD using the U-p53 peptide P1 is at least 94% in an asymptomatic individual. In some embodiments, the specificity of detection of progression of AD using the U-p53 peptide P1 is at least 95% in an asymptomatic individual. In some embodiments, the specificity of detection of progression of AD using the U-p53 peptide P1 is at least 96% in an asymptomatic individual.

In some embodiments, the specificity of detection of progression of AD using the U-p53 peptide P1 is at least 97% in an asymptomatic individual. Preferably, the specificity of detection of progression of AD using the U-p53 peptide P1 is at least 98% in an asymptomatic individual.

In some embodiments, the specificity of detection of progression of AD using the U-p53 peptide P1 is at least 70% in a subject suffering from MCI. In some embodiments, the specificity of detection of progression of AD using the U-p53 peptide P1 is at least 75% in a subject suffering from MCI. In some embodiments, the specificity of detection of progression of AD using the U-p53 peptide P1 is at least 80% in a subject suffering from MCI. In some embodiments, the specificity of detection of progression of AD using the U-p53 peptide P1 is at least 85% in a subject suffering from MCI. In some embodiments, the specificity of detection of progression of AD using the U-p53 peptide P1 is at least 90% in a subject suffering from MCI. In some embodiments, the specificity of detection of progression of AD using the U-p53 peptide P1 is at least 92% in a subject suffering from MCI.

In some embodiments, the specificity of detection of progression of AD using the U-p53 peptide P1 is at least 94% in a subject suffering from MCI. In some embodiments, the specificity of detection of progression of AD using the U-p53 peptide P1 is at least 95% in a subject suffering from MCI. In some embodiments, the specificity of detection of progression of AD using the U-p53 peptide P1 is at least 96% in a subject suffering from MCI. In some embodiments, the specificity of detection of progression of AD using the U-p53 peptide P1 is at least 97% in a subject suffering from MCI. Preferably, the specificity of detection of progression of AD using the U-p53 peptide P1 is at least 98% in a subject suffering from MCI.

In some embodiments, the method identifies a subject with at least 60% risk of progressing into Alzheimer's disease within the next 24 months of testing the biological sample of the subject. In some embodiments, the method identifies a subject with at least 70% risk of progressing into Alzheimer's disease within the next 24 months of testing the biological sample of the subject. In some embodiments, the method identifies a subject with at least 80% risk of progressing into Alzheimer's disease within the next 24 months of testing the biological sample of the subject. In some embodiments, the method identifies a subject with at least 85% risk of progressing into Alzheimer's disease within the next 24 months of testing the biological sample of the subject. In some embodiments, the method identifies a subject with at least 90% risk of progressing into Alzheimer's disease within the next 24 months of testing the biological sample of the subject. In some embodiments, the method identifies a subject with at least 92% risk of progressing into Alzheimer's disease within the next 24 months of testing the biological sample of the subject. In some embodiments, the method identifies a subject with at least 94% risk of progressing into Alzheimer's disease within the next 24 months of testing the biological sample of the subject. In some embodiments, the method identifies a subject with at least 95% risk of progressing into Alzheimer's disease within the next 24 months of testing the biological sample of the subject.

In some embodiments, the method identifies a subject with at least 60% risk of progressing into Alzheimer's disease within the next 6 years of testing the biological sample of the subject. In some embodiments, the method identifies a subject with at least 70% risk of progressing into Alzheimer's disease within the next 6 years of testing the biological sample of the subject. In some embodiments, the method identifies a subject with at least 80% risk of progressing into Alzheimer's disease within the next 6 years of testing the biological sample of the subject. In some embodiments, the method identifies a subject with at least 85% risk of progressing into Alzheimer's disease within the next 6 years of testing the biological sample of the subject. In some embodiments, the method identifies a subject with at least 90% risk of progressing into Alzheimer's disease within the next 6 years of testing the biological sample of the subject. In some embodiments, the method identifies a subject with at least 92% risk of progressing into Alzheimer's disease within the next 6 years of testing the biological sample of the subject. In some embodiments, the method identifies a subject with at least 94% risk of progressing into Alzheimer's disease within the next 6 years of testing the biological sample of the subject. In some embodiments, the method identifies a subject with at least 95% risk of progressing into Alzheimer's disease within the next 6 years of testing the biological sample of the subject.

In some embodiments, the method identifies subjects with at least 60% chance of not progressing into Alzheimer's disease within the next 2 years of testing the biological sample of the subject. In some embodiments, the method identifies a subject with at least 70% chance of not progressing into Alzheimer's disease within the next 2 years of testing the biological sample of the subject. In some embodiments, the method identifies a subject with at least 80% chance of not progressing into Alzheimer's disease within the next 2 years of testing the biological sample of the subject. In some embodiments, the method identifies a subject with at least 85% chance of not progressing into Alzheimer's disease within the next 2 years of testing the biological sample of the subject. In some embodiments, the method identifies a subject with at least 90% chance of not progressing into Alzheimer's disease within the next 2 years of testing the biological sample of the subject. In some embodiments, the method identifies a subject with at least 92% chance of not progressing into Alzheimer's disease within the next 2 years of testing the biological sample of the subject. In some embodiments, the method identifies a subject with at least 94% chance of not progressing into Alzheimer's disease within the next 2 years of testing the biological sample of the subject. In some embodiments, the method identifies a subject with at least 95% chance of not progressing into Alzheimer's disease within the next 2 years of testing the biological sample of the subject.

In some embodiments, the method identifies subjects with at least 60% chance of not progressing into Alzheimer's disease within the next 6 years of testing the biological sample of the subject. In some embodiments, the method identifies a subject with at least 70% chance of not progressing into Alzheimer's disease within the next 6 years of testing the biological sample of the subject. In some embodiments, the method identifies a subject with at least 80% chance of not progressing into Alzheimer's disease within the next 6 years of testing the biological sample of the subject. In some embodiments, the method identifies a subject with at least 85% chance of not progressing into Alzheimer's disease within the next 6 years of testing the biological sample of the subject. In some embodiments, the method identifies a subject with at least 90% chance of not progressing into Alzheimer's disease within the next 6 years of testing the biological sample of the subject. In some embodiments, the method identifies a subject with at least 92% chance of not progressing into Alzheimer's disease within the next 6 years of testing the biological sample of the subject. In some embodiments, the method identifies a subject with at least 94% chance of not progressing into Alzheimer's disease within the next 6 years of testing the biological sample of the subject. In some embodiments, the method identifies a subject with at least 95% chance of not progressing into Alzheimer's disease within the next 6 years of testing the biological sample of the subject.

In some embodiments, a subject affected by Alzheimer's disease, the area under the curve (AUC) is at least 75%. In a subject affected by Alzheimer's disease, the AUC is at least 80%. In a subject affected by Alzheimer's disease, the AUC is at least 85%. In a subject affected by Alzheimer's disease, the AUC is at least 90%. In a subject affected by Alzheimer's disease, the AUC is at least 95%.

A further embodiment of the present invention is a method of detecting the U-p53 peptide P1 as above described, the method comprising quantifying the U-p53 peptide P1 in a biological sample by comparing the same with a given control.

In a further preferred embodiment, the method of the present invention further comprising quantifying the amount of the U-p53 peptide P1 and determining the rate of progression of Alzheimer's disease.

In some embodiments of the method of the present invention, the quantity of the U-p53 peptide P1 is used to determine the rate of progression of Alzheimer's disease in asymptomatic individual and people suffering from MCI.

It should be also understood that all the combinations of preferred aspects of the peptides of the invention, as well as of the preparation processes, and methods using of the same, as above reported, are to be deemed as hereby disclosed.

### BRIEF DESCRIPTION OF FIGURES

FIG. 1 shows the schematic overview of diagnostic method for detecting the rate of progression of AD among subjects.

FIG. 2 shows the clinical validation cohort used for the study.

FIG. 3 shows the schematic representation of risk predictions based on the amount of U-p53 peptides in plasma sample.

FIG. 4 shows the graphical representation of concentration of AZ284 (P1 peptide) vs time graph which indicates that the diagnostic method identifies the quadrant of rapid progressors.

FIG. 5 shows the graphical representation of concentration of AZ284 (P1 peptide) vs time graph which indicates the ability of the diagnostic method to identify amyloid+ (amy+) progressors to AD in 2 years.

FIG. 6 discloses p53 protein sequence (SEQ ID NO: 16), the DNA binding domain of p53 protein is highlighted in grey, and the linear epitope (SEQ ID NO: 17) recognized by 2D3A8 antibody is underlined.

### DEFINITIONS

As used herein, the terms “unfolded’ or “conformational variant of or “misfolded’ or “unfolded/misfolded’ are used interchangeably. A protein is considered to be misfolded if it cannot achieve its native state of folding. Misfolding can be due to mutations in the amino acid sequence or a disruption of the normal folding process by external factors. A protein is considered to be unfolded (misfolded) when its native conformation is changed due to exposure to one or more of chemical, thermal or mechanical denaturation processes. Unfolded p53 and misfolded p53, are equivalent terms as used herein and refer to an unfolded isoform of the multifunctional protein p53 in plasma which has been known to be present in higher amounts in Alzheimer's Disease patients in comparison with healthy subjects. U-p53^{AZ} is an example of the unfolded/misfolded isoform of p53 protein which is bound by anti-p53 antibody such as 2D3A8. See Abate, G., Frisoni, G. B., Bourdon, J C. et al. The pleiotropic role of p53 in functional/dysfunctional neurons: focus on pathogenesis and diagnosis of Alzheimer's disease. Alz Res Therapy 12, 160.

As used herein, U-p53 peptides refer to peptides generated in vitro when the isolated U-p53 protein from the biological sample is digested with a protease, the protease preferably being trypsin. Preferably the unfolded p53 protein is isolated from the biological sample by immunoprecipitation using a suitable antibody specific for U-p53 protein such as 2D3A8. Some examples of U-p53 peptides include but not limited to P1 peptide, P2 peptide, P3 peptide and P4 peptide.

As used herein, “AZ284” refers to P1 peptide having sequence TEEENLR (SEQ ID N. 1) which is generated in vitro when the isolated U-p53 protein from the biological sample is digested with a protease such as trypsin.

As used herein, the term “sample” refers to a biological sample obtained from a subject, which could be any one of blood, saliva, cerebrospinal fluid, serum, and plasma.

As used herein, the term “rapid progressor” refers to an individual who has minimal or no symptoms of cognitive decline and has at least 1.06 fm, preferably at least 1.1 femtomoles, of U-p53 peptide detected in the biological sample. This individual will progress into AD within two years of testing.

As used herein, the term “high risk progressor” to an individual who has minimal or no symptoms of cognitive decline and has greater than 0.90 fm of U-p53 peptide detected in the biological sample. This individual is at a high risk of progressing into AD within 6 years of testing.

As used herein, the term “moderate progressor” or “not high risk progressor” or “excluding high risk progressor” refers to an individual who has minimal or no symptom of cognitive decline and has 0.75 to 0.90 fm (at least 0.75 fm but less than 0.90 fm) of U-p53 peptide detected in the biological sample. This individual has a moderate risk of progressing into AD within six years of testing.

As used herein, the term “slow progressor” or “low risk progressor” refers to an individual who has minimal or no symptom of cognitive decline and has less than 0.75 fm of U-p53 peptide detected in the biological sample. This individual has a low risk of progressing into AD within six years of testing.

As used herein, the term “risk of progression” refers to the probability or chance that the individual being tested will progress into (develop symptoms of) AD within a defined period of time.

As used herein, the term “high risk of progression” refers to the probability or chance that the individual being tested will progress into AD within 2-6 years from testing.

As used herein, the term “rapid progression” refers to the probability or chance that the individual being tested will progress into AD within 2 years from testing.

As used herein, the term “moderate risk of progression” or “not high risk of progression” or “excludes high risk of progression” refers to the probability or chance that the individual being tested will progress into AD within 6 years from testing. The subject having a moderate risk of progression or not high risk of progression are required and/or recommended to get tested every 6-12 months to monitor the level of U-p53 P1 peptide to assess their risk level and appropriate treatment options.

As used herein, the term “low risk of progression” refers to the probability or chance that the individual being tested will not progress into AD within 6 years from testing.

As used herein, the term “at least” refers to an amount or number that is equal to or greater than the designated amount or number. For example, at least 5 femtomoles includes 5 femtomoles and greater than 5 femtomoles such as 5.1 femtomoles, 5.15 fm, 5.2 fm etc.

As used herein, the term “less than” refers to an amount or number that is less than but not equal to the designated amount of number. For example, less than 5 femtomoles includes an amount that is lower than but not equal to 5 femtomoles such as 4.9 femtomoles, 4.85 fm, 4.8 fm etc.

As used herein, the term “greater than” refers to an amount or number that is greater than but not equal to the designated amount or number. For example, greater than 5 femtomoles includes an amount that is greater than but not equal to 5 femtomoles such as 5.1 femtomoles, 5.15 fm, 5.2 fm etc.

As used herein, the term “peptide control” refers to a known amount of peptide which serves as control for detection and quantification of signal during an assay such as mass spectrometry. Preferably the peptide control is a P1 peptide (TEEENLR) but it could be other peptides of p53 protein such as P2, P3 or P4 etc. In some instances, the peptide control is labelled so it can be distinguished from the sample and is referred to as labelled control peptide. In some instances, the control peptide is internal and a known amount of the peptide control is added to the sample prior to an assay such as mass spectrometry.

As used herein, the term “AUC” refers to Area under the receiver operating characteristic curve. A receiver operating characteristic curve, or ROC curve, is a graphical plot that illustrates the diagnostic ability of a binary classifier system as its discrimination threshold is varied. The ROC curve is created by plotting the true positive rate (TPR) against the false positive rate (FPR) at various threshold settings. The true-positive rate is also known as sensitivity, recall or probability of detection. The false-positive rate is also known as probability of false alarm and can be calculated as (1-specificity).

As used herein, the term “specificity” refers to (true negative rate) refers to the probability of a negative test, conditioned on truly being negative. Sensitivity and specificity mathematically describe the accuracy of a test which reports the presence or absence of a condition. Individuals for which the condition is satisfied are considered “positive” and those for which it is not considered “negative”.

As used herein, the term “sensitivity” refers to (true positive rate) refers to the probability of a positive test, conditioned on truly being positive.

As used herein, the term “cognitive normal (CN)” as individuals who are either asymptotic or exhibit minimal symptoms of cognitive decline.

As used herein the term “MCI” refers to a neurocognitive disorder which involves cognitive impairments beyond those expected based on an individual's age and education, but which are not significant enough to interfere with instrumental activities of daily living. MCI may occur as a transitional stage between normal aging and dementia, especially Alzheimer's disease.

As used herein the term “AD” refers to Alzheimer's disease (AD) which is a progressive neurodegenerative disorder, starting with an asymptomatic phase of normal cognition lasting approximately two decades. While some individuals experience subjective memory complaints (SMC), all eventually progress to mild cognitive impairment (MCI) before finally reaching the AD stage.

As used herein, the term “DMT” refers to disease-modifying treatment, disease-modifying drug, or disease-modifying therapy is a treatment that delays or slows the progression of a disease by targeting its underlying cause. They are distinguished from symptomatic treatments that treat the symptoms of a disease but do not address its underlying cause.

As used herein, the term “OD” refers to other forms of dementia.

As used herein, the term “Selected Reaction Monitoring (SRM)” refers to a method used in tandem mass spectrometry in which an ion of a particular mass is selected in the first stage of a tandem mass spectrometer and an ion product of a fragmentation reaction of the precursor ions is selected in the second mass spectrometer stage for detection. As used herein, the term “mini-mental state examination (MMSE)” refers to a 30-point questionnaire that is used extensively in clinical and research settings to measure cognitive impairment. It is commonly used in medicine and allied health to screen for dementia. It is also used to estimate the severity and progression of cognitive impairment and to follow the course of cognitive changes in an individual over time; thus, making it an effective way to document an individual's response to treatment. Scores on the MMSE range from 0 to 30, with scores of 26 or higher being traditionally considered cognitively normal. People with early-stage Alzheimer's disease tend to score in the 19 to 24 range and those with MCI score 25-26. (Arevalo-Rodriguez I, Smailagic N, Roqué I Figuls M, Ciapponi A, Sanchez-Perez E, Giannakou A, Pedraza O L, Bonfill Cosp X, Cullum S. Mini-Mental State Examination (MMSE) for the detection of Alzheimer's disease and other dementias in people with mild cognitive impairment (MCI). Cochrane Database Syst Rev. 2015 Mar. 5; 2015(3):CD010783)

As used herein, “symptoms of cognitive decline” include but are not limited to the following. A subject in cognitive decline will exhibit one or more of the following symptoms including memory loss, loss of focus, slow processing of facts and issues with complex decision making. A subject having AD will exhibit symptoms including memory loss, decline in non-memory aspects of cognition, such as finding the right word, trouble understanding visual images and spatial relationships, and impaired reasoning or judgment, getting lost and wandering, and difficulty in completing routine tasks such as bathing or eating.

As used herein, the term “memory loss” refers to the inability to remember facts, events, relationships including facts, information and experiences. Memory loss can be due to aging, and it can also occur due to neurodegenerative diseases such AD. Aging related memory loss is often characterized by the presence of common occurrences like (a) making a bad decision once in a while, (b) missing payments, (c) forgetting which day it is or forgetting the name of an acquaintance and (d) losing things from time to time. However, memory loss due to AD is characterized by the presence of unusual behaviour like (a) repeatedly exhibiting poor judgement and decision making, (b) inability to manage a budget, (c) losing track of date or month or season, (d) difficulty navigating path to work or home that were once well known and (e) difficulty having a conversation. Generally, memory loss due to AD begins to disrupt the subject's life. The subject often has trouble planning or solving problems, difficulty with completing familiar tasks such as brushing or eating or writing, trouble understanding visual images and spatial relationships, sudden changes in mood and personality.

### Signs of Memory Loss as a Part of Normal Aging

Inability to remember details of a conversation or event that took place a year ago.
Inability to remember the name of an acquaintance.
Tendency to forget things and events occasionally.
Occasional difficulty finding the right words.

### Signs of Memory Loss Due to AD

Inability to recall details of recent events or conversations.
Inability to recognize or know the names of family members.
Tendency to forget things or events more frequently.
Exhibition of frequent pauses and substitutions when finding words.

As used herein, “minimal symptoms of cognitive decline” refers to memory loss that occurs not due to aging and having trouble staying focused on task at hand.

As used herein, “no symptoms of cognitive decline” refers to having a MMSE score of 26 or higher, and not having any memory loss issues

As used herein, “detect”, “detecting” refers to identifying the presence or existence of U-p53 peptides in a reaction mixture generated by protease digestion of an immunoprecipitated biological sample from a subject.

As used herein, “quantify”, “quantifying” refers to measuring the amount or concentration of U-p53 peptides in a reaction mixture generated by protease digestion of an immunoprecipitated biological sample from a subject.

As used herein, “reaction mixture” refers to a composition that is generated when the immunoprecipitated biological sample from a subject is treated with a protease. The reaction mixture comprises proteolytic digestion products such as U-p53 peptides generated by the digestion of U-p53 protein by a protease such as trypsin.

As used herein, the term “ACE inhibitor” refers Angiotensin-converting enzyme inhibitors which inhibit ACE and help the body produce less angiotensin. ACE inhibitors help the blood vessels relax and open up, which, in turn, lowers blood pressure. Some non-limiting examples of ACE inhibitors include but not limited to benazepril hydrochloride, captopril, enalapril maleate, fosinopril sodium, lisinopril, moexipril, perindopril, quinapril hydrochloride and ramipril.

As used herein, the term “Angiotensin II receptor blocker” refers to drugs block the effects of angiotensin, a chemical that causes the arteries to become narrow. ARBs block the receptors, so the angiotensin fails to constrict the blood vessel resulting in decrease in blood pressure. Some non-limiting examples of Angiotensin II receptor blocker includes but not limited to candesartan, eprosartan mesylate, irbesartan, losartan potassium, telmisartan, and valsartan.

As used herein the term “beta blockers” refers to medications that reduce the heart rate, the heart's workload and the heart's output of blood, which lowers blood pressure. Examples include but not limited to acebutolol, atenolol, betaxolol, bisoprolol fumarate, carteolol hydrochloride, metoprolol tartrate, metoprolol succinate, nadolol, penbutolol sulfate and solotol hydrochloride.

As used herein, the term “calcium channel blocker” refers to drugs that prevents calcium from entering the smooth muscle cells of the heart and arteries. When calcium enters these cells, it causes a stronger and harder contraction, so by decreasing the calcium, the hearts' contraction is not as forceful. Calcium channel blockers relax and broaden narrowed blood vessels, reduce heart rate and lower blood pressure. Some non-limiting examples of calcium channel blocker include but not limited to amlodipine besylate, bepridil, diltiazem hydrochloride, felodipine, isradipine, nicardipine, nifedipine, nisoldipine and verapamil hydrochloride.

As used herein, the term “DASH” diet refers to Dietary Approaches to Stop Hypertension diet. The DASH diet is a healthy-eating plan designed to help treat or prevent high blood pressure (hypertension). The DASH diet includes foods that are rich in potassium, calcium, and magnesium. These nutrients help control blood pressure. The diet limits foods that are high in sodium, saturated fat and added sugars. Studies have shown that the DASH diet can lower blood pressure in as little as two weeks. The diet can also lower low-density lipoprotein (LDL or “bad”) cholesterol levels in the blood. High blood pressure and high LDL cholesterol levels are two major risk factors for heart disease and stroke. The DASH diet is rich in vegetables, fruits, and whole grains. It includes fat-free or low-fat dairy products, fish, poultry, beans, and nuts. It limits foods that are high in saturated fat, such as fatty meats and full-fat dairy products. (Appel L J, Moore T J, Obarzanek E, Vollmer W M, Svetkey L P, Sacks F M, Bray G A, Vogt T M, Cutler J A, Windhauser M M, Lin P H, Karanja N. A clinical trial of the effects of dietary patterns on blood pressure. DASH Collaborative Research Group. N Engl J Med. 1997 Apr. 17; 336(16):1117-24)

As used herein the term “MIND” diet refers to Mediterranean-Dash Intervention for Neurodegenerative Delay (MIND) diet. It is a hybrid of the Mediterranean and Dash diets, with modifications based on the science of nutrition and the brain. MIND diet typically encourages fruits, vegetables, whole grains, legumes, nuts, seeds, and heart-healthy fats. Processed foods, added sugar, and refined grains should be restricted. MIND diet has been shown to be effective in preventing cognitive decline after stroke. (Cherian L, Wang Y, Fakuda K, Leurgans S, Aggarwal N, Morris M. Mediterranean-Dash Intervention for Neurodegenerative Delay (MIND) Diet Slows Cognitive Decline After Stroke. J Prev Alzheimers Dis. 2019; 6(4):267-273. doi: 10.14283/jpad.2019.28.)

As used herein the term “fmol” refers to femtomoles of U-p53 peptide.

### Treatment

A subject that is classified as high risk (rapid progressor) of progressing to AD within 2 years, moderate risk (not being at high risk), or low risk (slow progressor) is then treated by a medical professional according to acceptable medical standards. A subject determined to be at high risk or not at high risk but at moderate risk and possibly some low-risk subjects may be determined by a skilled medical profession to require a certain treatment regimen. For example, a skilled medical professional may determine that a disease-modifying treatment, disease-modifying drug, or disease-modifying therapy is applicable to a given subject. Such a treatment may delay or slow the progression of a disease by targeting its underlying cause.

In some embodiments, the method is useful in stratifying participants in early AD clinical trials for measuring effectiveness of novel drugs for AD treatment. In some embodiments, the method is useful for triaging subjects in clinical settings on risk-benefit basis for treatment with AD drugs. In some embodiments, the method is useful for individuals to plan for scenarios such as DNR, Will and advanced care treatments.

In some embodiments, the method is useful in recruiting participants in primary prevention studies (e.g. AHEAD, DIAN) who may have the brain pathology of AD (confirmed by amyloid and tau testing) but are not at a high-risk for immediate deterioration. In some embodiments, the method is useful for triaging subjects in clinical settings and to identify individuals who are at risk for developing AD by monitoring the level of U-p53 peptides at regular intervals such as 6-12 months. In some embodiments, the method is useful for individuals who have family history of dementia or AD to test their personal risk profile.

In some embodiments, the method further comprises the step of treating the subject classified as one of high risk, moderate risk, or low risk of progression to AD.

In some embodiments of any of the aforesaid methods, the subject is classified as high risk progressor if the U-p53-P1 peptide is present in concentrations greater than 0.90 femtomoles then the subject is classified as a high risk progressor of AD. A high risk progressor is at high risk of progressing into AD within the next 6 years. The subject thus classified as a high risk progressor could be then treated with one or more disease modifying medications selected from the group consisting of aducanumab and lecanemab.

In some embodiments of any of the aforesaid methods, the subject is classified as a rapid progressor if the U-p53-P1 peptide is present in concentrations at least 1.06 femtomoles, preferably at least 1.1 femtomoles. A rapid progressor is at high risk of progressing into AD within 2 years. The subject thus classified as a high risk progressor could be then treated with one or more disease modifying medications selected from the group consisting of aducanumab and lecanemab.

In some embodiments of any of the aforesaid methods, the subject, 6-12 months post treatment is tested again following the same procedures outlined above to see if the concentration of U-p53-P1 peptide is increased or decreased or stayed the same.

In some embodiments of any of the aforesaid methods, if the concentration of U-p53-P1 peptide increases post treatment for a high risk progressor, then the dosage and/or frequency of administration is increased as it might imply the drug is not effective at the dosage being administered. If the concentration of U-p53-P1 peptide decreases post treatment for a high risk progressor, then the drug being administered is considered to be effective in treating AD and the treatment regimen is maintained. If the concentration of U-p53-P1 peptide remains unchanged post treatment for a high risk progressor, then the drug being administered is considered to be effective at preventing the progression of AD and optionally the drug dosage is incrementally increased to determine optimal dose for the subject.

In some embodiments of any of the aforesaid methods, the subject is classified as a moderate progressor or excluding high risk risk progressor if the U-p53-P1 peptide is present in concentrations greater than 0.75 femtomoles and less than 0.90 femtomoles then the subject is classified as a moderate risk progressor of AD. A moderate risk progressor is at moderate risk of progressing into AD within the next 6 years. The subject thus classified as a moderate risk progressor is then treated with one or more drugs selected from the group consisting of memantine galantamine, rivastigmine, and donepezil.

In some embodiments of any of the aforesaid methods, the subject, 6-12 months post treatment is tested again following the same procedures outlined above to see if the concentration of U-p53-P1 peptide is increased or decreased or stayed the same.

In some embodiments of any of the aforesaid methods, if the concentration of U-p53-P1 peptide increases post treatment, then the dosage and/or frequency of administration is increased as it might imply that the drug is not effective at the dosage being administered. If the concentration of U-p53-P1 peptide decreases post treatment, then the drug being administered is considered to be effective in treating AD and the treatment regimen is maintained. If the concentration of U-p53-P1 peptide remains unchanged post treatment, then the drug being administered is considered to be effective at preventing the progression of AD and optionally the drug dosage is incrementally increased to determine optimal dose for the subject.

In some embodiments of any of the aforesaid methods, the subject is classified as a slow progressor or a low risk progressor if the U-p53-P1 peptide is present in concentrations less than 0.75 femtomoles then the subject is classified as a low risk progressor of AD. A low risk progressor is at low risk of progressing into AD within the next 6 years. The subject thus classified as a low risk progressor is then treated with one or more MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

In some embodiments of any of the aforesaid methods, the subject thus classified as a low risk progressor is then treated with one or more MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

In some embodiments of any of the aforesaid methods, the vitamin/antioxidant supplement is one or more of vitamin A, vitamin B, folic acid, vitamin C, vitamin D, vitamin E, DHA (docosahexaenoic acid), ubiquinone, lycopene, coenzyme Q10 and ellagic acid, ascorbic acid, masoprocol, pramipexole, nitric oxide, allopurinol, pentoxifylline, melatonin, probucol, quercetin, acetylcysteine, n acetylcysteine, acetyl-L-carnitine and 1-methylfolate and resveratrol. (Mielech A, Puścion-Jakubik A, Markiewicz-Żukowska R, Socha K. Vitamins in Alzheimer's Disease-Review of the Latest Reports. Nutrients. 2020 Nov. 11; 12(11):3458).

In some embodiments of any of the aforesaid methods, the blood pressure reducing medication is selected from the group consisting of Angiotensin-converting enzyme (ACE) inhibitors, Angiotensin receptor blockers (ARBs), Calcium-channel blockers and Beta-blockers.

In some embodiments of any of the aforesaid methods, the anti-inflammatory medication is selected from the group consisting of Aspirin, Diclofenac, Etodolac, Fenoprofen, Flurbiprofen, Ibuprofen, Indomethacin, Meclofenamate, Mefenamic Acid, Nabumetone, Naproxen, Oxaprozin, Piroxicam, Sulindac, Tolmetin, Celecoxib and Meloxicam.

In some embodiments of any of the aforesaid methods, the subject, 6-12 months post treatment is tested again following the same procedures outlined above to see if the concentration of U-p53-P1 peptide is increased or decreased or stayed the same.

In some embodiments of any of the aforesaid methods, if the concentration of U-p53-P1 peptide increases post treatment, then the treatment regimen is not effective and is discontinued. If the concentration of U-p53-P1 peptide decreases post treatment, then the treatment regimen being administered is considered to be effective in preventing AD and the treatment regimen is maintained. If the concentration of U-p53-P1 peptide remains unchanged post treatment, then the drug being administered is considered to be effective at preventing the progression of AD and optionally the additional lifestyle changes such as physical exercise can be added to the treatment regimen.

All combinations of the preferred aspects of the peptides of the invention, preparation processes, and methods disclosed above are to be understood as herein described. Below are working examples of the present invention provided for illustrative purposes.

```
SEQUENCES
U-p53 peptide-P1
SEQ ID NO: 1
TEEENLR
U-p53 peptide-P2
SEQ ID NO: 2
TEEENLRK[GG]K
U-p53 peptide-P3
SEQ ID NO: 3
KKPLDGEYFTLQIR
U-p53 peptide-P4
SEQ ID NO: 4
EPGGSRAHSSHLK
U-p5 peptide-P5
SEQ ID NO: 5
GEPHHELPPGSTKRALPNNTSSSPQPK
Heavy chain of 2D3A8 antibody
SEQ ID NO: 6
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser Glu Ser Gln Ser Phe Pro Asn Val Phe Pro Leu
                115 120 125
Val Ser Cys Glu Ser Pro Leu Ser Asp Lys Asn Leu Val Ala Met Gly
        130 135 140
Cys Leu Ala Arg Asp Phe Leu Pro Ser Thr Ile Ser Phe Thr Trp Asn
145 150 155 160
Tyr Gln Asn Asn Thr Glu Val Ile Gln Gly Ile Arg Thr Phe Pro Thr
                                165 170 175
Leu Arg Thr Gly Gly Lys Tyr Leu Ala Thr Ser Gln Val Leu Leu Ser
                        180 185 190
Pro Lys Ser Ile Leu Glu Gly Ser Asp Glu Tyr Leu Val Cys Lys Ile
                195 200 205
His Tyr Gly Gly Lys Asn Arg Asp Leu His Val Pro Ile Pro Ala Val
        210 215 220
Ala Glu Met Asn Pro Asn Val Asn Val Phe Val Pro Pro Arg Asp Gly
225 230 235 240
Phe Ser Gly Pro Ala Pro Arg Lys Ser Lys Leu Ile Cys Glu Ala Thr
                                245 250 255
Asn Phe Thr Pro Lys Pro Ile Thr Val Ser Trp Leu Lys Asp Gly Lys
                        260 265 270
Leu Val Glu Ser Gly Phe Thr Thr Asp Pro Val Thr Ile Glu Asn Lys
                275 280 285
Gly Ser Thr Pro Gln Thr Tyr Lys Val Ile Ser Thr Leu Thr Ile Ser
        290 295 300
Glu Ile Asp Trp Leu Asn Leu Asn Val Tyr Thr Cys Arg Val Asp His
305 310 315 320
Arg Gly Leu Thr Phe Leu Lys Asn Val Ser Ser Thr Cys Ala Ala Ser
                                325 330 335
Pro Ser Thr Asp Ile Leu Thr Phe Thr Ile Pro Pro Ser Phe Ala Asp
                        340 345 350
Ile Phe Leu Ser Lys Ser Ala Asn Leu Thr Cys Leu Val Ser Asn Leu
                355 360 365
Ala Thr Tyr Glu Thr Leu Asn Ile Ser Trp Ala Ser Gln Ser Gly Glu
        370 375 380
Pro Leu Glu Thr Lys Ile Lys Ile Met Glu Ser His Pro Asn Gly Thr
385 390 395 400
Phe Ser Ala Lys Gly Val Ala Ser Val Cys Val Glu Asp Trp Asn Asn
                                405 410 415
Arg Lys Glu Phe Val Cys Thr Val Thr His Arg Asp Leu Pro Ser Pro
                        420 425 430
Gln Lys Lys Phe Ile Ser Lys Pro Asn Glu Val His Lys His Pro Pro
                435 440 445
Ala Val Tyr Leu Leu Pro Pro Ala Arg Glu Gln Leu Asn Leu Arg Glu
        450 455 460
Ser Ala Thr Val Thr Cys Leu Val Lys Gly Phe Ser Pro Ala Asp Ile
465 470 475 480
Ser Val Gln Trp Leu Gln Arg Gly Gln Leu Leu Pro Gln Glu Lys Tyr
                                485 490 495
Val Thr Ser Ala Pro Met Pro Glu Pro Gly Ala Pro Gly Phe Tyr Phe
                        500 505 510
Thr His Ser Ile Leu Thr Val Thr Glu Glu Glu Trp Asn Ser Gly Glu
                515 520 525
Thr Tyr Thr Cys Val Val Gly His Glu Ala Leu Pro His Leu Val Thr
        530 535 540
Glu Arg Thr Val Asp Lys Ser Thr Gly Lys Pro Thr Leu Tyr Asn Val
545 550 555 560
Ser Leu Ile Met Ser Asp Thr Gly Gly Thr Cys Tyr
                                565 570
Light chain of 2D3A8 antibody
SEQ ID NO: 7
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys Arg Ala Asp Ala Ala
                        100 105 110
Pro Thr Val Ser Ile Phe Pro Pro Ser Ser Glu Gln Leu Thr Ser Gly
                115 120 125
Gly Ala Ser Val Val Cys Phe Leu Asn Asn Phe Tyr Pro Lys Asp Ile
        130 135 140
Asn Val Lys Trp Lys Ile Asp Gly Ser Glu Arg Gln Asn Gly Val Leu
145 150 155 160
Asn Ser Trp Thr Asp Gln Asp Ser Lys Asp Ser Thr Tyr Ser Met Ser
                                165 170 175
Ser Thr Leu Thr Leu Thr Lys Asp Glu Tyr Glu Arg His Asn Ser Tyr
                        180 185 190
Thr Cys Glu Ala Thr His Lys Thr Ser Thr Ser Pro Ile Val Lys Ser
                195 200 205
Phe Asn Arg Asn Glu Cys
        210
Heavy chain variable region of 2D3A8 antibody
SEQ ID NO: 8
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser
                115
Light chain variable region of 2D3A8 antibody
SEQ ID NO: 9
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys
                        100 105
Heavy chain CDR1 of 2D3A8 antibody
SEQ ID NO: 10
Ser Tyr Val Met His
1 5
Heavy chain CDR2 of 2D3A8 antibody
SEQ ID NO: 11
Tyr Ile Asn Pro Tyr Asn Asp Gly Thr Lys Tyr Asn Glu Lys Phe Lys
1 5 10 15
Gly
Heavy chain CDR3 of 2D3A8 antibody
SEQ ID NO: 12
Gly Gly Tyr Tyr Ala Met Asp Tyr
1 5
Light chain CDR1 of 2D3A8 antibody
SEQ ID NO: 13
Arg Ala Ser Gln Asp Ile Ser Asn Tyr Leu Asn
1 5 10
Light chain CDR2 of 2D3A8 antibody
SEQ ID NO: 14
Tyr Thr Ser Arg Leu His Ser
Light chain CDR3 of 2D3A8 antibody
SEQ ID NO: 15
Gln Gln Gly Asn Thr Leu Pro Tyr Thr
p53 Protein
SEQ ID NO: 16
Met Glu Glu Pro Gln Ser Asp Pro Ser Val Glu Pro Pro Leu Ser Gln
1 5 10 15
Glu Thr Phe Ser Asp Leu Trp Lys Leu Leu Pro Glu Asn Asn Val Leu
                        20 25 30
Ser Pro Leu Pro Ser Gln Ala Met Asp Asp Leu Met Leu Ser Pro Asp
                35 40 45
Asp Ile Glu Gln Trp Phe Thr Glu Asp Pro Gly Pro Asp Glu Ala Pro
        50 55 60
Arg Met Pro Glu Ala Ala Pro Pro Val Ala Pro Ala Pro Ala Ala Pro
65 70 75 80
Thr Pro Ala Ala Pro Ala Pro Ala Pro Ser Trp Pro Leu Ser Ser Ser
                                85 90 95
Val Pro Ser Gln Lys Thr Tyr Gln Gly Ser Tyr Gly Phe Arg Leu Gly
                        100 105 110
Phe Leu His Ser Gly Thr Ala Lys Ser Val Thr Cys Thr Tyr Ser Pro
                115 120 125
Ala Leu Asn Lys Met Phe Cys Gln Leu Ala Lys Thr Cys Pro Val Gln
        130 135 140
Leu Trp Val Asp Ser Thr Pro Pro Pro Gly Thr Arg Val Arg Ala Ala
145 150 155 160
Ile Tyr Lys Gln Ser Gln His Met Thr Glu Val Val Arg Arg Cys Pro
                                165 170 175
His His Glu Arg Cys Ser Asp Ser Asp Gly Leu Ala Pro Pro Gln His
                        180 185 190
Leu Ile Arg Val Glu Gly Asn Leu Arg Val Glu Tyr Leu Asp Asp Arg
                195 200 205
Asn Thr Phe Arg His Ser Val Val Val Pro Tyr Glu Pro Pro Glu Val
        210 215 220
Gly Ser Asp Cys Thr Thr Ile His Tyr Asn Tyr Met Cys Asn Ser Ser
225 230 235 240
Cys Met Gly Gly Met Asn Arg Arg Pro Ile Leu Thr Ile Ile Thr Leu
                                245 250 255
Glu Asp Ser Ser Gly Asn Leu Leu Gly Arg Asn Ser Phe Glu Val Arg
                        260 265 270
Val Cys Ala Cys Pro Gly Arg Asp Arg Arg Thr Glu Glu Glu Asn Leu
                275 280 285
Arg Lys Lys Gly Glu Pro His His Glu Leu Pro Pro Gly Ser Thr Lys
        290 295 300
Arg Ala Leu Pro Asn Asn Thr Ser Ser Ser Pro Gln Pro Lys Lys Lys
305 310 315 320
Pro Leu Asp Gly Glu Tyr Phe Thr Leu Gln Ile Arg Gly Arg Glu Arg
                                325 330 335
Phe Glu Met Phe Arg Glu Leu Asn Glu Ala Leu Glu Leu Lys Asp Ala
                        340 345 350
Gln Ala Gly Lys Glu Pro Gly Gly Ser Arg Ala His Ser Ser His Leu
                355 360 365
Lys Ser Lys Lys Gly Gln Ser Thr Ser Arg His Lys Lys Leu Met Phe
        370 375 380
Lys Thr Glu Gly Pro Asp Ser Asp
385 390
Linear Epitope
SEQ ID NO: 17
Arg Arg Thr Glu Glu Glu Asn Leu Arg Lys Lys Gly Glu Pro His His
1 5 10 15
```

### EXAMPLES

### Materials and Methods

### Subjects

The plasma samples and supporting clinical information were provided by the Australian Imaging, Biomarkers and Lifestyle (AIBL) longitudinal cohort study, Subjects aged between 60 and 85 years who did not present specific comorbidities (uncontrolled diabetes, vascular disease, severe depression, or psychiatric illnesses) were included in this retrospective study, blood samples were collected for each included subject from the study by applying a consecutive sampling approach.

Subjects were followed-up every 18 months. At each visit, the neuropsychological status and medical history were reviewed by a neuropsychologist and clinician. A clinical review panel consisting of a geriatrician, a neurologist, and a neuropsychologist, blinded to amyloid β-positron emission tomography (Aβ-PET) and biomarker status, determined the diagnosis of CN, MCI and AD subjects.

Data on the mini-mental state examination (MMSE), clinical dementia rating Scale and amyloid brain burden (determined by PET with the labelled Pittsburgh compound B [PiB-PET], flutemetamol, florbetapir, or NAV4694) were collected following established protocols (Klunk W E, Koeppe R A, Price J C, et al. The Centiloid Project: Standardizing quantitative amyloid plaque estimation by PET. Alzheimer's & Dementia 2015; 11, 1-15.e4; Piccirella S, Van Neste L, Fowler C, Masters C L, Fripp J, Doecke J D, Xiong C, Uberti D, Kinnon P. A Conformational Variant of p53 (U-p53AZ) as Blood-Based Biomarker for the Prediction of the Onset of Symptomatic Alzheimer's Disease. J Prev Alzheimers Dis. 2022; 9(3):469-479)

### Blood Processing and APOE Alleles Genotyping

According to the AIBL standard operating procedures, blood samples were handled at room temperature in EDTA collection tubes (S-Monovette, Sarstedt, Germany) preventively supplemented with prostaglandin-E (final concentration: 33 ng/ml, Sapphire Biosciences, Australia). Processing took place within three hours from blood withdrawal, followed by aliquoted sample storage in liquid nitrogen. Genotyping was carried out as has been previously described (Gupta V B, Laws S M, Villemagne V L, et al. Plasma apolipoprotein E and Alzheimer disease risk: the AIBL study of aging. Neurology 2011; 76, 1091-1098).

### Immunoprecipitation (IP) and Nanoflow Electrospray Ionization Tandem Mass Spectrometry (ESI-MS/MS)

The 2D3A8 antibody selectively binds the AD-clinically relevant U-p53AZ protein and was used to identify peptides through protein sequencing via MS/MS. Protein sequencing was performed at MyomicsDX Inc. (MD, USA). Immunoprecipitation (IP) took place on high abundance protein-depleted plasma samples with different antibodies. One reaction was performed with 2D3A8 (10 μg/sample, Diadem SpA, Italy), while the other was based on a mixture of p53-specific antibodies at 10 μg/sample (DO₁₁:DO₁₂:SAPU:KJC₁₂, respective volume ratios 1:1:2:2 and final concentration of 1 μg/μl).

Upon enrichment, the peptides were eluted, fractionated through an Agilent 1290 Infinity II liquid chromatography (LC) system (Agilent, CA, USA) and analyzed in MS by a Thermo Scientific™ Q Exactive Mass Spectrometer (Thermo Fisher Scientific, MA, USA). Tandem mass tag labelled peptides were analyzed by MS/MS in a data-dependent approach on a Thermo Scientific™ EASY-nLC 1000™ HPLC system, coupled to a Thermo Scientific EASYSpray™ source supported by an analytical nanoflow column system (Thermo Fisher Scientific, MA, USA).

Acquisition of the survey full scan MS spectra (m/z 350-1800) was achieved with the Orbitrap with 35,000 resolution, following ion accumulation to a 3×106 target value, selected on predictive automated gain control based on the previous full scan. Sequential isolation of the 10 most intense multiply charged ions (z≥2) followed and these were then fragmented in the Axial Higher energy Collision-induced Dissociation (HCD) cell through normalized HCD collision energy at 30% (automatic gain control target: 1e5, maximal injection time: 400 ms, resolution: 35,000).

The Proteome Discoverer 2.2 software (Thermo Fisher Scientific, MA, USA) processed automatically the MS raw files, and Xtract was deployed in addition to default spectrum selector node. The Mascot search engine combined with Sequest HT (interfaced with different processing nodes of Proteome Discoverer 2.2) was used to address the searches. The final dataset was reprocessed through the MyProt-QuantiR (MyOmicsDx Inc., MD, USA) software package, allowing the identification of the AZ 284® peptide sequence as the most clinically relevant peptide in samples from individuals affected by AD.

### Sample Preparation

Fractions of samples provided by the AIBL cohort (25 μL) underwent IP using the patented 2D3A8 monoclonal antibody (30) (Diadem SpA, Italy) coupled to Protein L magnetic beads (Thermo Fisher Scientific, MA, USA). Upon protein enrichment, the samples were treated with trypsin (3.5 hours at 37° C. followed by 0.5 hours at 57° C.).

Measurement of U-p53 Peptides in Patient Reaction Mixture Generated from Plasma Sample

U-p53 plasma levels were assessed through quantization of the peptide AZ 284® (i.e., U-p53-P1 peptide) by LC coupled to electrospray ionization (ESI)-MS/MS at ISB srl (Italy). A surveyor HPLC Thermofisher Quaternary Pump was paired to a ThermoFisher mass spectrometer. For analyte separation, a Phenomenex Kinetex PFP column (50×4.1 mm, 2.6 m, Phenomenex, CA, USA) was used and the mobile phases were A (H₂O-0.2% HCOOH, Sigma-Aldrich, MO, USA) and C (CH3OH, Sigma-Aldrich, MO, USA). LC was performed in a binary gradient at a chromatographic flow of 0.2 mL/min: 2% of phase C was maintained for 2 minutes and raised to 40% in 3 minutes and maintained as such for 7 additional minutes. Phase C was further raised to 70% 2 minutes later and kept at 70% for 4 minutes before resetting to the starting conditions for 5 minutes.

A calibration curve was developed prior to analysis of the clinical samples, spiking a negative control of plasma with AZ 284® labelled peptide covering the range between 0.02 fmol/10 μl and 4 fmol/10 μl by at least 4 different points. Peptide sequence was then analyzed with a ThermoFisher Mass Spectrometer (TSQ Vantage Thermo Fisher Scientific heated-ESI ion source, MA, USA). The ion source parameters were multiple reaction monitoring scan; capillary temperature: 320° C.; collision energy: 5 eV; sheath gas flow: 30 L/min; auxiliary gas flow: 2 L/min; and sweep gas flow: 15 L/min. All tests were performed while being blinded from the clinical and cognitive data.

### Statistical Analyses

The diagnostic and prognostic performance of U-p53 peptides to predict the rate of progression of AD were assessed. The performance of amyloid status (as measured by AB imaging, calibrated CL, or inferred Aβ categories as reported above) was included as reference at the diagnostic level and compared with U-p53 peptides at the prognostic level. Survival curves were plotted on risk models based on U-p53 peptide alone or a model encompassing all the risk factors. Diagnostic analyses were performed on the biomarker data and neuropsychological assessments and the analyses used measurements defined at the baseline assessment (i.e., the sample corresponding to the visit at which U-p53 was first measured). The diagnostic performance was evaluated through two specific models based on the neuropsychological assessment: 1) comparing AD individuals with non-AD individuals (including subjects in the study pooled from any other diagnosis than AD) and 2) specifically comparing CN and MCI subjects with AD subjects. For prognostic evaluation, both time-independent and time-dependent analyses were performed to verify the potential of U-p53-P1 in predicting the progression of AD. Time-independent analyses were structured using the baseline measure of U-p53 peptide and AB-PET to discern AD-progressing individuals from stable non-AD subjects based on their final neuropsychological diagnosis. Time-dependent analyses of individuals regardless of the neuropsychologically assigned status (AD versus non-AD) were included and compared when available with information on amyloid status. In a sub-cohort of samples with available amyloid status data, the prognostic performance of U-p53 peptide was compared to the performance of the AB-PET biomarker. In this comparison, non-AD individuals for which their true disease status was unknown or ambiguous due to lack of sufficient follow-up were right-censored in these specific analyses. A survival analysis with respect of AD-free survival was performed excluding subjects with a baseline neuropsychological AD diagnosis to assess the prognostic value of U-p53 peptide from the baseline measurement. A right-censored model was considered to be sufficiently accurate and allowed the use of a semi-parametric Cox proportional hazards model, based on 1) the considerably larger median follow-up times for CN (67 months), MCI (27 months) and OD (37 months) compared to the interval period between patient visits (approximately 18 months) during which AD onset could occur, and 2) the 20-month median time to progression observed for those patients whose neuropsychological status changed to AD.

Cox models were evaluated for 1) U-p53 peptide alone or 2) along with other covariates (U-p53AZ, CL, baseline age, gender and dichotomous APOE ε4 status). Hazard ratios (HR) were then evaluated for all risk factors in models adjusted or unadjusted to the factors and two models (U-p53AZ and a complete model including all factors) were then plotted as Kaplan-Meier curves to visualize the probability of AD onset according to the risk strata. The analyses were obtained using survival, survminer, and survcomp R packages. For direct HR comparison, the continuous variables of the reference model (U-p53AZ, AB-PET [CL] and age) were normalized into standard scores by subtracting the mean from each value and dividing by the standard deviation. To evaluate diagnostic and prognostic performances, receiver operating characteristic (ROC) curves were generated and their corresponding area under the curve (AUC) were calculated to determine the accuracy, sensitivity, specificity, positive (PPV) and negative predictive values (NPV), using the R-package bdpv.

Unless otherwise specified, cut-offs for sensitivity and specificity were determined using the maximal Youden J statistic, i.e., the sum of sensitivity and specificity. Prognostic analyses were performed in R, using the pROC and timeROC packages for time-independent and time-dependent analyses respectively (Blanche P, Dartigues J, Jacqmin-Gadda H. Estimating and Comparing time dependent areas under receiver operating characteristic curves for censored event times with competing risks. Statistics in Medicine, 2013; 32, 5381-5397; Robin X, Turck N, Hainard A, et al. pROC: an open-source package for R and S+ to analyze and compare ROC curves. BMC Bioinformatics 2011; 12, 77 and R Core Team, 2020. R: A language and environment for statistical computing. R Foundation for Statistical Computing, Vienna, Austria. URL https://www.R-project.org/).

ROC curve analyses to determine the optimal cut-off were performed using the cutpointr package (Thiele C and Hirschfeld G. cutpointr: Improved Estimation and Validation of Optimal Cutpoints in R. J Stat Softw 2021; 98). Where applicable, numerical values were compared using Student's T test or ANOVA followed by Tukey's test for evaluation of pairwise differences. Differences in follow-up time were evaluated using a non-parametric Wilcoxon rank sum test with continuity correction. Fisher's exact test was used to evaluate categorical count data. Comparison of ROC curves were performed through the DeLong test, while NPV and PPV were defined at a prevalence rate of AD of 30% at baseline and 45% at the final diagnosis. All the mentioned analyses were carried out in R package.

### Correlation Between p53 Sequence Peptides and Rate of Progression of AD.

### Example 1—Validation of Use of U-p53 Peptides for Determining the Rate of Progression of Alzheimer's Disease at the Pre-Clinical and Prodromal Stages of the Disease

Initial results from a longitudinal four-year study on clinical progression of cognitively normal subjects and people with mild cognitive impairment to probable AD measuring U-p53 signals measured by direct ELISA has shown very high predictive values for progression. A highly accurate mass spectrometry method based on the identification of U-p53 peptide-P1 for the determination of rate of progression of Alzheimer's disease and for the prognosis of cognitive decline was developed to determine the rate of progression of AD in subjects.

The maximum prognostic PPV (Predictive positive value) for cognitive decline to Alzheimer's disease was achieved in both subjects with MCI and asymptomatic. A specific mass spec method known as the SRM (Selected Monitoring Reaction) method (by triple quadrupole mass spectrometer) was used to quantify P1 in a reaction mixture generated from immunoprecipitated plasma samples from AD, asymptomatic, MCI subjects by protease digestion. The triple quadrupole mass spectrometer acts as essentially as a mass filter allows the sequencing of the U-p53 peptide Pls being generated by looking at the different transition peaks. Then, using a heavy labelled internal control for peptide 1, it is possible to quantitate those peptides with the maximum precision by Selected Monitoring Reaction (SRM) method.

### Prognostic Performance

The prognostic power of the biomarker in enriching for patients who are PiB+ but also will exhibit cognitive decline into AD in 24-60 months for asymptomatic subjects at presentation. This was performed in order to assess the value of the biomarker in significantly enriching for cognitive decline as an endpoint in disease modifying trials in asymptomatic and patients with MCI. The prognostic power of P1 was determined using the future conversion of clinical presentation. Statistical parameters able to describe the prognostic power of P1 are below reported:

TABLE 1
Prognostic Power of Asymptomatic subjects for predicting
high risk of progression within 6 years

| | Sequence |
|---|---|
| Parameters | peptide 1 |
| Sensitivity | 82.4% |
| Specificity | 98.0% |
| PPV (positive predictive | 91.8% |
| value) | |
| NPV (negative predictive | 95.3% |
| value) | |
| Prevalence (AD Progression) | 21.5% |
| Risk zone | 5.7% |

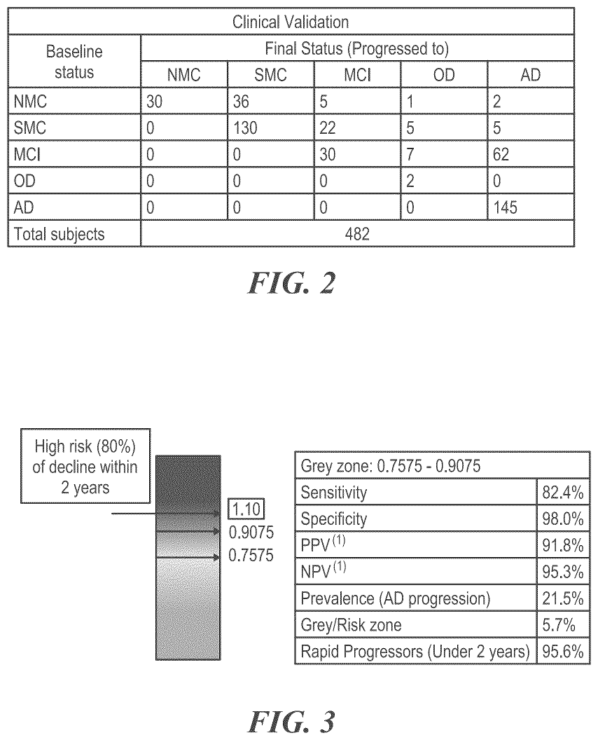

A higher PPV translates into a lower number of false-positive results. Test performance calculated by counting intermediate results as low-risk, actual numbers will be based on ADRC and ADNI data added to AIBL data sets. See FIGS. 2 and 3.

TABLE 2
Prognostic Power of Asymptomatic subjects for predicting
high risk of progression within 2 years

| | Sequence |
|---|---|
| Parameters | peptide 1 |
| Sensitivity | 47.8% |
| Specificity | 99.6% |
| PPV (positive predictive | 97% |
| value) | |
| NPV (negative predictive | 87.5% |
| value) | |
| Prevalence (AD | 21.5% |
| progression) | |
| Rapid Progressors (Under 2 | 95.6% |
| years) | |

TABLE 3
Prognostic Power of Asymptomatic subjects for predicting high risk of
progression within 2 years corelated with presence of amyloids. The diagnostic method is
able to identify 100% of progressors to AD/amyloid positive (amy +ve) 2 years in advance
before the occurrence of cognitive decline.

| | All Pts | | | | Max 2 years (for AD Dx, all controls) | | | |
|---|---|---|---|---|---|---|---|---|
| AZ284 | Amy +ve* | Amy +ve* | Amy +ve % | Amy +VE % | Amy +ve* | Amy +ve* | Amy +ve % | Amy +VE % |
| 0.9 | 27 | 2 | 93.10% | 6.90% | 19 | 2 | 90.48% | 9.52% |
| 0.9075 | 27 | 2 | 93% | 7% | 19 | 2 | 90.48% | 9.52% |
| (high end | | | | | | | | |
| prognostic cutoff | | | | | | | | |
| 1 | 20 | 1 | 95.24% | 4.76% | 15 | 1 | 93.75% | 6.25% |
| 1.065 | 11 | 0 | 100.00% | 0.00% | 8 | 0 | 100.00% | 0.00% |
| 1.0675 | 11 | 0 | 100.00% | 0.00% | 8 | 0 | 100.00% | 0.00% |
| 1.1 | 5 | 0 | 100.00% | 0.00% | 5 | 0 | 100.00% | 0.00% |
| 1.2 | 1 | 0 | 100.00% | 0.00% | 1 | 0 | 100.00% | 0.00% |
| 1.3 | 1 | 0 | 100.00% | 0.00% | 1 | 0 | 100.00% | 0.00% |
| 1.4 | 0 | 0 | NA | NA | 0 | 0 | NA | NA |
| 1.5 | 0 | 0 | NA | NA | 0 | 0 | NA | NA |

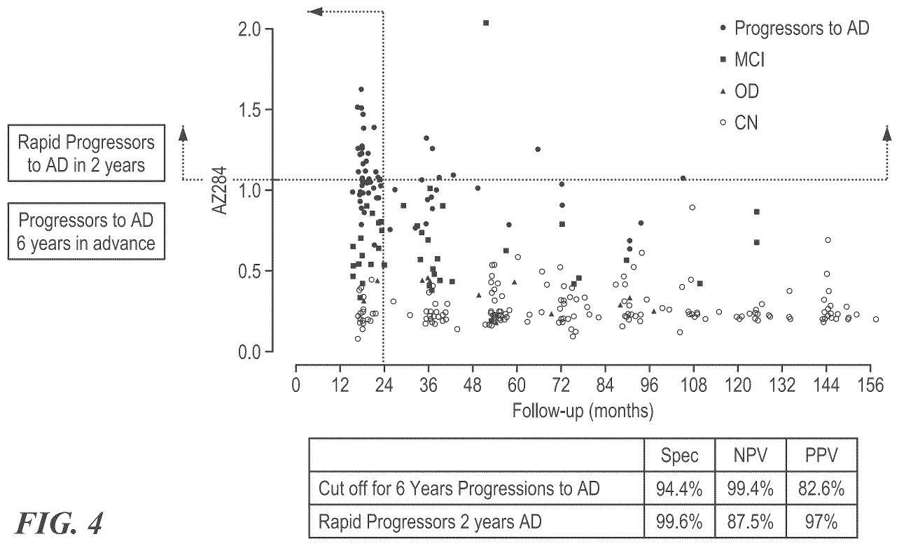

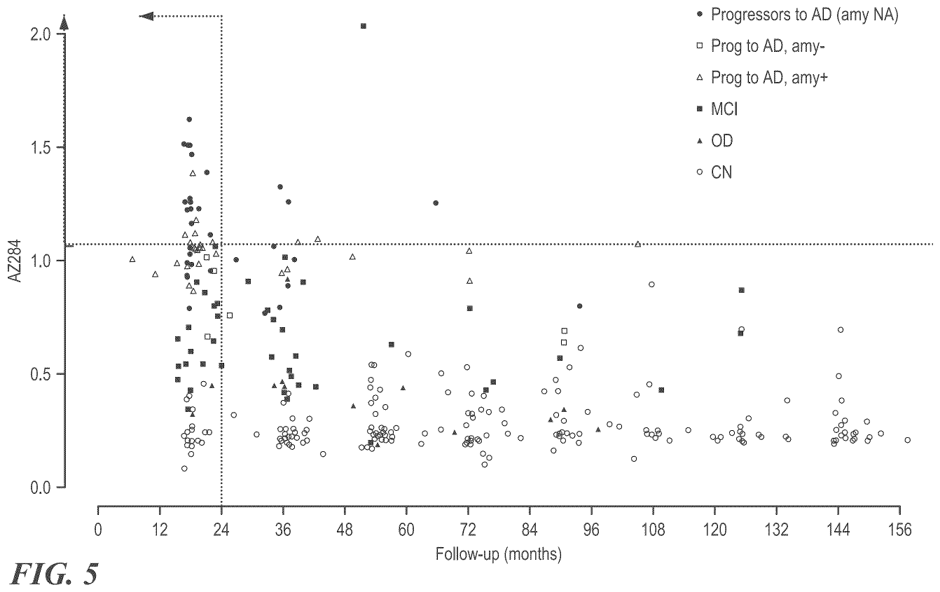

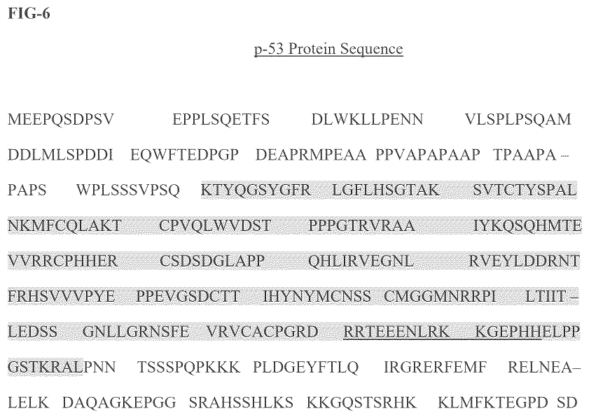

The diagnostic method thus allows one to identify from a cohort as to which individuals will rapidly progress into AD within two years of testing. The method thus enables the successful recruitment of patients for disease modifying drugs targeting the very early stages of the disease. For early-AD studies, 2 years' time frame is the clinical trial time horizon, ensuring that only those, who are at risk to deteriorated (rapid progressors) during the study, will be recruited. See FIGS. 3-5.

Likewise, the method can also be used for identifying suitable candidates for primary-prevention studies (e.g., AHEAD (https://www.aheadstudy.org/), Dominantly Inherited Alzheimer Network (DIAN)) since the test can enable the selection of individuals, who may have the brain pathology of AD (confirmed by amyloid and tau testing) but are not at a high-risk for immediate deterioration.

The method can also be used by clinicians to perform risk-benefit analysis to determine which individuals need to be treated. The decision as to whether treat individuals who are already at the cusp of dementia or those who are still far away from accelerated deterioration. Knowing the level of risk, combined with the overall status of the patient can help drive the decision-making, to become patient focused. The method also can assist medical professionals for triaging subjects in clinical settings and to identify individuals who are at risk for developing AD by continuously monitoring the level of U-p53 peptides.

The method can also be used for monitoring the efficacy of a candidate AD treatment drug by monitoring the level of U-p53 peptides in subjects at regular intervals (examples of time intervals for repeat testing include but not limited to 6-12 months or 3-6 months or 1-2 months, every 2 weeks, every week) and determine if the drug being administered is able to prevent the progression of AD in subjects with high risk for progression.

The method can also be useful for individuals to plan in advance for scenarios such as DNR, Will, trusts and advanced care treatments by determining whether they are rapid progressors and plan their lives accordingly. The method can also be useful for individuals who have family history of dementia or AD to determine their personal risk profile with respect to rapid or moderate or slow progression of disease state.

### Example 2—Diagnosis and Treatment of High Risk Progressor

The example provides a method of identifying a high risk progressor and treating the progressor with a suitable treatment that may include medication for treating AD. The method can also be used to classify patients as high risk progressors for recruiting candidates for clinical trials aimed at treating severe stages of AD.

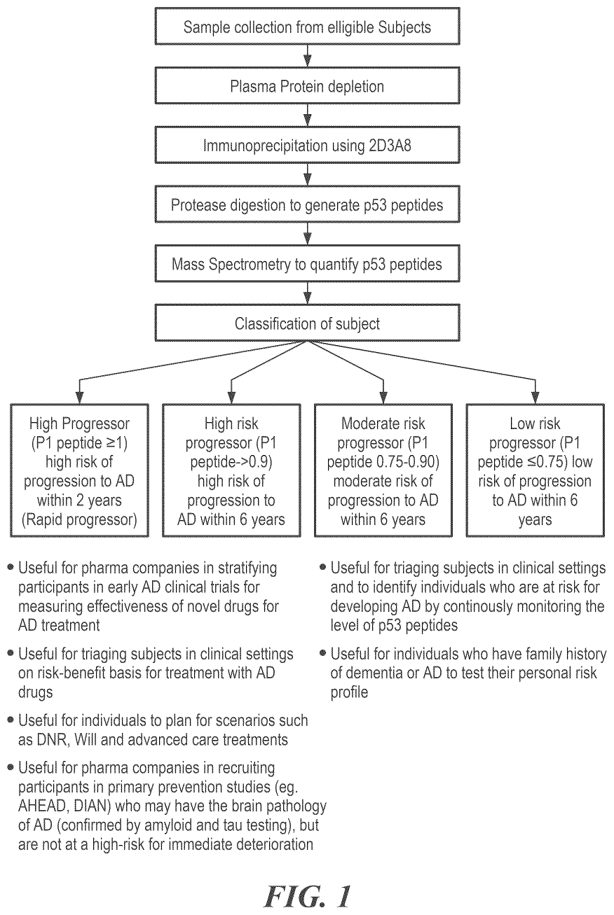

A biological sample is first obtained from the subject. The sample can be one of blood, saliva, CSF, plasma, or serum. The sample is then subject to processing as show in FIG. 1 following the procedures as outlined above. Briefly the sample is subject to Plasma Protein depletion followed by immunoprecipitation by 2D3A8 antibody, the immunoprecipitate is then subjected to proteolytic digest by trypsin. The reaction mixture thus generated is subjected to HPLC and Mass spectrometry to determine the identity and quantity of each U-p53 peptide generated by the proteolysis. The amount of U-p53-P1 peptide is calculated.

A subject that is classified as high risk (rapid progressor) of progressing to AD within 2 years or a high risk of progressing to AD within 6 years is then treated by a medical professional according to acceptable medical standards. A subject determined to be at high risk may be determined by a skilled medical profession to require a certain treatment regimen. For example, a skilled medical professional may determine that a disease-modifying treatment, disease-modifying drug, or disease-modifying therapy is applicable to a given subject. Such a treatment may delay or slow the progression of a disease by targeting its underlying cause.

If the p53-P1 peptide is present in concentrations greater than 0.90 femtomoles then the subject is classified as a high risk progressor of AD. A high risk progressor is at high risk of progressing into AD within the next 6 years. If the U-p53-P1 peptide is present in concentrations at least 1.06 femtomoles, preferably at least 1.1 femtomoles, then the subject is classified as a rapid progressor of AD. A rapid progressor is at high risk of progressing into AD within the next 2 years.

The subject thus classified as a rapid progressor or high risk progressor may be then treated with one or more disease modifying medications selected from the group consisting of aducanumab and lecanemab. The subject, six months post treatment is tested again following the same procedures outlined above to see if the concentration of U-p53-P1 peptide increases or decreases. Treatment is considered and determined by a skilled medical professional.

If the concentration of U-p53-P1 peptide increases post treatment, then a skilled medical professional may determine that the dosage and/or frequency of administration is increased, as an increase in P1 peptide post-treatment may indicate that a given drug is not effective at the dosage being administered. If the concentration of U-p53-P1 peptide decreases post treatment, then the drug being administered may be considered to be effective in treating AD and the treatment regimen may be maintained, optionally the drug dosage is incrementally increased to determine optimal dose for the subject. If the concentration of p53-P1 peptide remains unchanged post treatment, then the drug being administered may be considered to be effective at preventing the progression of AD and optionally the drug dosage is incrementally increased to determine optimal dose for the subject.

### Example 3—Diagnosis and Treatment of Moderate Risk Progressor

The example provides a method of identifying a moderate risk progressor and treating the progressor with suitable medications for treating early-stage AD. The method can also be used to classify patients as moderate risk progressors for recruiting candidates for clinical trials aimed at treating or preventing early stages of AD such as AHEAD or DIAN trials.

A biological sample is first obtained from the subject. The sample can be one of blood, saliva, CSF, plasma, or serum. The sample is then subject to processing as show in FIG. 1 following the procedures as outlined above in Example 2. Briefly the sample is subject to Plasma Protein depletion followed by immunoprecipitation by 2D3A8 antibody, the immunoprecipitated is then subjected to proteolytic digest by trypsin. The reaction mixture thus generated is subjected to HPLC and Mass spectrometry to determine the identity and quantity of each U-p53 peptide generated by the proteolysis. The amount of p53-P1 peptide is calculated.

A subject that is classified as moderate risk (not at high risk for progression to AD) is then treated by a medical professional according to acceptable medical standards. A subject determined to be at not at high risk but at moderate risk may be determined by a skilled medical profession to require a certain treatment regimen. For example, a skilled medical professional may determine that a disease-modifying treatment, disease-modifying drug, or disease-modifying therapy is applicable to a given subject. Such a treatment may delay or slow the progression of a disease by targeting its underlying cause.

If the U-p53-P1 peptide is present in concentrations greater than or equal to 0.75 femtomoles (at least 0.75 fm) and less than 0.90 femtomoles then the subject is classified as a moderate risk progressor of AD. A moderate risk progressor is at moderate risk of progressing into AD within the next 6 years. The subject thus classified as a moderate risk progressor is then treated with one or more drugs selected from the group consisting of memantine galantamine, rivastigmine, and donepezil. The subject, six months post treatment is tested again following the same procedures outlined above to see if the concentration of U-p53-P1 peptide increases or decreases.

If the concentration of U-p53-P1 peptide increases post treatment, then the dosage and/or frequency of administration may be determined by a medical profession to require an increase, an increase in P1 peptide may indicate a given drug is not effective at the dosage being administered. If the concentration of U-p53-P1 peptide decreases post treatment, then the drug being administered may be considered to be effective in treating AD and the treatment regimen may be maintained, optionally the drug dosage is incrementally increased to determine optimal dose for the subject. If the concentration of U-p53-P1 peptide remains unchanged post treatment, then the drug being administered is considered to be effective at preventing the progression of AD and optionally the drug dosage is incrementally increased to determine optimal dose for the subject.

### Example 4—Diagnosis and Treatment of Low Risk Progressor

The example provides a method of identifying low risk progressor and treating the progressor with suitable treatment options to prevent the occurrence of AD. The method can also be useful for individuals who have no symptoms of AD but have family history of AD or dementia. The method thus allows those individuals to determine their personal risk of getting AD and steps that they can take suitable steps (exercise, healthy diet, lowering inflammation, reducing blood pressure, and lowering cardiovascular risks) to reduce the risk of getting AD.

A biological sample is first obtained from the subject. The sample can be one of blood, saliva, CSF, plasma, or serum. The sample is then subject to processing as show in FIG. 1 following the procedures as outlined above. Briefly the sample is subject to Plasma Protein depletion followed by immunoprecipitation by 2D3A8 antibody, the immunoprecipitated is then subjected to proteolytic digest by Trypsin. The reaction mixture thus generated is subjected to HPLC and Mass spectrometry to determine the identity and quantity of each U-p53 peptide generated by the proteolysis. The amount of U-p53-P1 peptide is calculated.

A subject that is classified as low risk (slow progressor) is then treated by a medical professional according to acceptable medical standards. A subject determined to be low risk subjects may be determined by a skilled medical profession to require a certain treatment and/or diet regimen. For example, a skilled medical professional may determine that a specific diet or supplement is applicable to a given subject. Such a treatment may delay or slow the progression of a disease by targeting its underlying cause.

If the U-p53-P1 peptide is present in concentrations less than 0.75 femtomoles then the subject is classified as a low risk progressor of AD. A low risk progressor is at low risk of progressing into AD within the next 6 years. The subject thus classified as a low risk progressor is then treated with one or more MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

The vitamin/antioxidant supplement is one or more of vitamin A, vitamin B, folic acid, vitamin C, vitamin D, vitamin E, DHA (docosahexaenoic acid), ubiquinone, lycopene, coenzyme Q10 and ellagic acid, ascorbic acid, masoprocol, pramipexole, nitric oxide, allopurinol, pentoxifylline, melatonin, probucol, quercetin, acetylcysteine, n acetylcysteine, acetyl-L-carnitine and 1-methylfolate and resveratrol. (Mielech A, Puścion-Jakubik A, Markiewicz-Żukowska R, Socha K. Vitamins in Alzheimer's Disease-Review of the Latest Reports. Nutrients. 2020 Nov. 11; 12(11):3458)

The blood pressure reducing medication is selected from the group consisting of Angiotensin-converting enzyme (ACE) inhibitors, Angiotensin receptor blockers (ARBs), Calcium-channel blockers and Beta-blockers.

The anti-inflammatory medication is selected from the group consisting of Aspirin, Diclofenac, Etodolac, Fenoprofen, Flurbiprofen, Ibuprofen, Indomethacin, Meclofenamate, Mefenamic Acid, Nabumetone, Naproxen, Oxaprozin, Piroxicam, Sulindac, Tolmetin, Celecoxib and Meloxicam.

The subject, 6-12 months post treatment is tested again following the same procedures outlined above to see if the concentration of U-p53-P1 peptide is increased or decreased or stayed the same.

If the concentration of U-p53-P1 peptide increases post treatment, then the treatment regimen is not effective and is discontinued.

If the concentration of U-p53-P1 peptide decreases post treatment, then the treatment regimen being administered is considered to be effective in preventing AD and the treatment regimen is maintained.

If the concentration of U-p53-P1 peptide remains unchanged post treatment, then the drug being administered is considered to be effective at preventing the progression of AD and optionally the additional lifestyle changes such as physical exercise can be added to the treatment regimen. (Meng Q, Lin M S, Tzeng I S. Relationship Between Exercise and Alzheimer's Disease: A Narrative Literature Review. Front Neurosci. 2020 Mar. 26; 14:131.)

### REFERENCES

1. Stanga, S. et al., 2010. Unfolded p53 in the pathogenesis of Alzheimer's disease: Is HIPK2 the link? Aging, 2(9), pp. 545-554.
2. Lanni, C. et al., 2007. Unfolded p53: A potential biomarker for Alzheimer's disease. In Journal of Alzheimer's Disease. pp. 93-99.
3. Uberti, D. et al., 2008. Conformationally altered p53: a putative peripheral marker for Alzheimer's disease. Neuro-degenerative diseases, 5(3-4), pp. 209-11.
4. Lanni, C. et al., 2008. Conformationally altered p53: a novel Alzheimer's disease marker? Molecular psychiatry, 13(6), pp. 641-7.
5. Lanni, C., Racchi, M., et al., 2010. Unfolded p53 in blood as a predictive signature signature of the transition from mild cognitive impairment to Alzheimer's disease. Journal of Alzheimer's disease: JAD, 20(1), pp. 97-104.
6. Buizza, L. et al., 2012. Conformational altered p53 as an early marker of oxidative stress in Alzheimer's disease. PloS one, 7(1), p.e29789
7. Arce-Varas N, et al. Comparison of extracellular and intracellular blood compartments highlights redox alterations in Alzheimer's and Mild Cognitive Impairment patients. Current Alzheimer Research 2017; 14(1): 112-122.
8. Uberti, D. et al., 2006. Identification of a mutant-like conformation of p53 in fibroblasts from sporadic Alzheimer's disease patients. Neurobiology of aging, 27(9), pp. 1193-201.
9. Lanni, C., Nardinocchi, L., et al., 2010. Homeodomain interacting protein kinase 2: a target for Alzheimer's beta amyloid leading to misfolded p53 and inappropriate cell survival. PloS one, 5(4), p.e10171.
10. Lanni, C. et al., 2008. Pharmacogenetics and Pharmagenomics, Trends in Normal and Pathological Aging Studies: Focus on p53. Current Pharmaceutical Design, 14(26), pp. 2665-2671.

### INCORPORATION BY REFERENCE

The disclosure of each and every U.S. and foreign patent and pending patent application and publication referred to herein is specifically incorporated herein by reference in its entirety, as are the contents of Figures. All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

### EQUIVALENTS

Those skilled in the art will recognize or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### OTHER EMBODIMENTS

From the foregoing description, it will be apparent that variations and modifications may be made to the disclosure described herein to adopt it to various usages and conditions within the scope according to the disclosure. Other embodiments according to the disclosure are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

Other embodiments are within the following claims.

1-61. (canceled)

62. A method of classifying a subject as having a risk level selected from high risk, moderate risk, rapid progression, low risk or slow progression to Alzheimer's disease, the method comprising:
subjecting a biological sample from said subject to immunoprecipitation using an antibody specific for U-p53 protein to provide isolated U-p53 protein;
subjecting said isolated U-p53 protein to protease digestion to generate one or more proteolytic peptides, wherein said one or more proteolytic peptides comprises peptide P1 having SEQ ID NO: 1 TEEENLR; and
quantifying an amount of said P1 peptide in a reaction mixture,
wherein:
at least 1.06 femtomoles indicates rapid progression;
at least 0.9 femtomoles indicates high risk progression;
at least 0.75 but less than 0.9 femtomoles indicates moderate progression or excludes high risk; and
less than 0.75 femtomoles indicates low risk or slow progression,
wherein said amount corresponds to said risk level.

63. A method of making a reaction mixture for classifying a subject for risk of progression to Alzheimer's disease (AD), the method comprising:
subjecting a biological sample from said subject to immunoprecipitation using an antibody specific for p53 protein to provide isolated U-p53 protein;
subjecting said isolated U-p53 protein to protease digestion to generate peptide P1 having SEQ ID NO:1; and
forming a reaction mixture comprising said peptide P1,
wherein said antibody comprises a heavy chain variable region comprising CDR1 (SEQ ID NO: 10), CDR2 (SEQ ID NO:11) and CDR3 (SEQ ID NO:12) and a light chain variable region comprising CDR1 (SEQ ID NO:13), CDR2 (SEQ ID NO:14) and CDR3 (SEQ ID NO:15),
wherein said reaction mixture comprises an amount of said peptide P1 that:
is at least 1.06 femtomoles, indicating rapid progression to AD;
is at least 0.9 femtomoles, indicating high risk of progression to AD;
is at least 0.75 femtomoles but less than 0.9 femtomoles, indicating moderate progression or exclusion of high risk; or
is less than 0.75 femtomoles, indicating low risk or slow progression to AD.

64. The method of claim 62, further comprising the step of administering to said subject a therapeutic intervention selected from a disease-modifying anti-amyloid therapy, a cognitive symptom-modifying therapy, a dietary intervention, a vitamin or antioxidant supplement, a blood pressure reducing medication, or an anti-inflammatory medication appropriate for said risk level.

65. A reaction mixture for assessing risk of progression to Alzheimer's disease (AD), comprising peptide P1 having SEQ ID NO:1, wherein said peptide P1 is present in an amount selected from:
at least 1.06 femtomoles;
at least 0.9 femtomoles;
at least 0.75 femtomoles but less than 0.9 femtomoles; or
less than 0.75 femtomoles,
wherein said amount corresponds to rapid progression, high risk progression, moderate progression or exclusion of high risk, or low risk or slow progression to AD, respectively.

66. The method of claim 62, wherein said quantifying is performed using mass spectrometry and said mass spectrometry comprises HPLC-mass spectrometry.

67. The method of claim 62, wherein quantifying is performed using Selected Reaction Monitoring (SRM).

68. The method of claim 67, wherein SRM identifies and quantifies said P1 peptide by comparison with a peptide control value.

69. The method of claim 62, wherein said biological sample is selected from blood, serum, plasma, saliva and cerebrospinal fluid.

70. The method of claim 69, wherein said biological sample is plasma.

71. The method of claim 70, wherein said plasma is subjected to protein depletion prior to immunoprecipitation.

72. The method of claim 71, wherein said protein depletion is accomplished by one or more of HPLC, a chromatographic column, or chemical treatment.

73. The method of claim 62, wherein said method provides prognosis within 2-6 years in an asymptomatic subject or a subject exhibiting mild cognitive impairment.

74. The method of claim 62, wherein said antibody comprises a heavy chain variable region having SEQ ID NO:8 and a light chain variable region having SEQ ID NO:9.

75. The method of claim 62, wherein said antibody comprises a heavy chain having SEQ ID NO: 6 and a light chain having SEQ ID NO:7.

76. The method of claim 64, wherein said disease-modifying anti-amyloid therapy comprises aducanumab or lecanemab.

77. The method of claim 64, wherein said cognitive symptom-modifying therapy is selected from memantine, galantamine, rivastigmine, and donepezil.

78. The method of claim 64, wherein said dietary intervention comprises a MIND diet or DASH diet.

79. The method of claim 64, wherein said vitamin or antioxidant supplement is selected from vitamin A, vitamin B, folic acid, vitamin C, vitamin D, vitamin E, DHA, ubiquinone, coenzyme Q10, ellagic acid, ascorbic acid, melatonin, resveratrol, acetyl-L-carnitine, and 1-methylfolate.

80. The method of claim 64, wherein said blood pressure reducing medication is selected from ACE inhibitors, ARBs, calcium channel blockers, and beta blockers.

81. The method of claim 64, wherein said anti-inflammatory medication is selected from aspirin, diclofenac, etodolac, fenoprofen, flurbiprofen, ibuprofen, indomethacin, meclofenamate, mefenamic acid, nabumetone, naproxen, oxaprozin, piroxicam, sulindac, tolmetin, celecoxib, and meloxicam.


SEQUENCE LISTING (17 sequences)

SEQ ID NO: 1 - protein, 7 aa, synthetic construct
  REGION 1..7: note = U-p53 peptide-P1
TEEENLR

SEQ ID NO: 2 - protein, 11 aa, synthetic construct
  REGION 1..11: note = U-p53 peptide-P2
TEEENLRKGGK

SEQ ID NO: 3 - protein, 14 aa, synthetic construct
  REGION 1..14: note = U-p53 peptide-P3
KKPLDGEYFTLQIR

SEQ ID NO: 4 - protein, 13 aa, synthetic construct
  REGION 1..13: note = U-p53 peptide-P4
EPGGSRAHSSHLK

SEQ ID NO: 5 - protein, 27 aa, synthetic construct
  REGION 1..27: note = U-p5 peptide-P5
GEPHHELPPGSTKRALPNNTSSSPQPK

SEQ ID NO: 6 - protein, 572 aa, synthetic construct
  REGION 1..572: note = Heavy chain of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 7 - protein, 214 aa, synthetic construct
  REGION 1..214: note = Light chain of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 117 aa, synthetic construct
  REGION 1..117: note = Heavy chain variable region of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 9 - protein, 107 aa, synthetic construct
  REGION 1..107: note = Light chain variable region of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 10 - protein, 5 aa, synthetic construct
  REGION 1..5: note = Heavy chain CDR1 of 2D3A8 antibody
SYVMH

SEQ ID NO: 11 - protein, 17 aa, synthetic construct
  REGION 1..17: note = Heavy chain CDR2 of 2D3A8 antibody
YINPYNDGTKYNEKFKG

SEQ ID NO: 12 - protein, 8 aa, synthetic construct
  REGION 1..8: note = Heavy chain CDR3 of 2D3A8 antibody
GGYYAMDY

SEQ ID NO: 13 - protein, 11 aa, synthetic construct
  REGION 1..11: note = Light chain CDR1 of 2D3A8 antibody
RASQDISNYLN

SEQ ID NO: 14 - protein, 7 aa, synthetic construct
  REGION 1..7: note = Light chain CDR2 of 2D3A8 antibody
YTSRLHS

SEQ ID NO: 15 - protein, 9 aa, synthetic construct
  REGION 1..9: note = Light chain CDR3 of 2D3A8 antibody
QQGNTLPYT

SEQ ID NO: 16 - protein, 392 aa, Homo sapiens
  REGION 1..392: note = p53 Protein
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 17 - protein, 16 aa, synthetic construct
  REGION 1..16: note = Linear Epitope
RRTEEENLRKKGEPHH